(19) United States
(12) Patent Application Publication
(10) Pub. No.: US 2014/0256931 A1
(43) Pub. Date: Sep. 11, 2014

(54) CARBONACEOUS MATERIAL FOR PURIFYING LIGNOCELLULOSIC OLIGOMERS

(71) Applicants: The Regents of the University of California, Oakland, CA (US); BP Corporation North American Inc., Houston, TX (US)

(72) Inventors: Po-Wen Chung, El Cerrito, CA (US); Alexandre Charmot, Berkeley, CA (US); Alexander Katz, El Sobrante, CA (US); Amit A. Gokhal, El Cerrito, CA (US)

(73) Assignees: BP CORPORATION NORTH AMERICA INC., Houston, TX (US); THE REGENTS OF THE UNIVERSITY OF CALIFORNIA, Oakland, CA (US)

(21) Appl. No.: 14/352,684

(22) PCT Filed: Oct. 18, 2012
(86) PCT No.: PCT/US2012/060911

§ 371 Date: Apr. 17, 2014

Related U.S. Application Data
(60) Provisional application No. 61/548,685, filed on Oct. 18, 2011.

(51) Int. Cl. C07H 1/06; C07H 3/04; C07H 3/06; C07H 3/02

(52) U.S. Cl. CPC C07H 1/06; C07H 3/02; C07H 3/04; C07H 3/06
USPC 536/123.1; 536/127; 536/123.13; 536/123.12

(57) ABSTRACT

The present disclosure relates carbonaceous materials and to methods of using such carbonaceous materials for purifying oligomers produced from depolymerized biomass, such as lignocellulosic biomass.

### CROSS-REFERENCE TO RELATED APPLICATIONS

This application claims the benefit of U.S. Provisional Patent Application Ser. No. 61/548,685, filed Oct. 18, 2011, which is incorporated herein by reference in its entirety.

### FIELD

The present disclosure relates to methods and compositions for purifying oligomers, such as oligosaccharides, from depolymerized biomass, such as lignocellulosic biomass.

### BACKGROUND

Lignocellulosic biomass is an abundant renewable resource and a potential feedstock for the production of biofuels and other commodity chemicals. In the case of ethanol-based biofuels, all of the historic cost reductions reported from 1979 to 1986 have resulted from improvements in lignocellulose depolymerization and pretreatment, such as acid hydrolysis of hemicellulose (Wyman E. C. Annu. Rev. Energy Environ., 1999, 24: 189-226). In other words, all of the historic cost reductions resulted from improvements in the ability to convert lignocellulose from biomass into fermentable sugars.

One main obstacle to fermenting raw lignocellulosic biomass is the inaccessibility of the poly-β-glucan chains contained in the cellulose fraction of lignocellulosic biomass. A solution to this problem is to transform the tightly packed poly-β-glucan chains in cellulose into accessible oligosaccharides, which can be readily fermented by hydrolysis. One method for converting cellulosic poly-β-glucan chains into oligosaccharides is the Arkenol process described in U.S. Pat. No. 5,726,046. The Arkenol process utilizes concentrated aqueous acid to hydrolyze the poly-β-glucan chains and release the resulting oligosaccharides into solution, as well as a polymer resin to adsorb the released oligosaccharides, which allows the concentrated aqueous acid to be recycled. However, the adsorbent used by the Arkenol process to separate the oligosaccharides from concentrated acid is a cation exchange resin that does not have a high porosity. It would thus be advantageous to utilize an adsorbent that has a greater porosity and affinity for oligosaccharides, such as glucose, in the presence of concentrated aqueous acid.

An alternative approach for adsorbing oligosaccharides during the hydrolysis of lignocellulose is to use carbon-based materials for the adsorption of oligosaccharides. For example, activated carbon has been previously shown to have significant capacity for glucose at neutral pH (Bui et al., Ind. Eng. Chem. Process Des. Dev. 1985, 24, 1209-1213; and Blum et al. Archives of Biochemistry and Biophysics 1960, 91, 21-26). However, these carbon materials have not been demonstrated to adsorb longer chain oligosaccharides, nor has it been reported that such carbon materials are adsorbent at acidic pH values.

Accordingly, a need exists for improved carbon-based materials for adsorbing oligosaccharides during depolymerization, such as hydrolysis, of lignocellulose that have an increased affinity and capacity for both long chain and short chain oligosaccharides, and that function at acidic pH values.

### BRIEF SUMMARY

In order to meet the above needs, the present disclosure provides novel methods of purifying oligomers, such as oligosaccharides, from depolymerized biomass, such as lignocellulosic biomass, by utilizing carbonaceous material, such as mesoporous carbon material, to adsorb the oligomers, and novel functionalized mesoporous carbon materials containing phosphonic acid for depolymerizing lignocellulosic biomass. In some embodiments, the method further includes desorbing the adsorbed oligomers from the MCM.

In some embodiments, the providing of an oligomer-containing solution includes: providing biomass; and depolymerizing the biomass to produce an oligomer-containing solution. In certain embodiments, the biomass is lignocellulosic biomass. In other embodiments, the biomass contains cellulose. In still other embodiments, the biomass contains hemicellulose. In further embodiments, the biomass is cellulosic biomass.

In some embodiments of any of the above methods, the oligomer-containing solution contains lignin oligomers. In other embodiments of any of the above methods, the oligomer-containing solution contains hemicellulose oligomers. Preferably, the hemicellulose oligomers are oligosaccharides. In certain embodiments, the oligosaccharides are selected from xylose, xylans, xyloglucans, mannans, mannose, galactose, rhamnose, arabinose, arabinoxylans, and combinations thereof.

In yet other embodiments of any of the above methods, the oligomer-containing solution contains poly-β-glucan fragments derived from cellulose. Preferably, the poly-β-glucan fragments contain oligosaccharides. In certain embodiments, the oligosaccharides are selected from glucose, cellobiose, cellotriose, cellotetraose, cellopentaose, cellohexaose, and combinations thereof.

In further embodiments of any of the above methods, the oligomer-containing solution contains long chain poly-β-glucans.

In some embodiments of any of the above methods, the depolymerizing includes contacting the biomass with water at a temperature above at least 50° C.

In other embodiments of any of the above methods, the depolymerizing includes acid hydrolysis. In certain embodiments, the depolymerizing includes contacting the solution with an acid. In some embodiments, the acid is a dilute acid. In other embodiments, the acid is a concentrated acid. In still other embodiment, the acid is a mineral acid. Preferably, the mineral acid is HCl, H₃PO₃, H₃PO₄, H₂SO₄, H₂SO₃, HF, HlO₄, HBr, H₃O⁺, HNO₂, HNO₃, HI, boronic acid, or polyoxometallate acid. In yet other embodiments, the acid is an organic acid. Preferably, the organic acid is acetic acid or carboxylic acid.

In some embodiments of any of the above methods, the depolymerizing includes alkaline hydrolysis. In certain embodiments, the depolymerizing includes contacting the solution with a base. Preferably, the base is dilute ammonia, NH₄OH, NAOH, KOH, or LiOH.

In other embodiments of any of the above methods, the depolymerizing includes contacting the biomass with hot water, one or more organic acids, one or more supercritical fluids, one or more near-supercritical fluids, or combinations thereof. In still other embodiments of any of the above methods, the depolymerizing includes contacting the biomass with one or more ionic liquids. In certain embodiments, the contacting occurs at a temperature above about 50° C. In yet other embodiments of any of the above methods, the depolymerizing includes contacting the biomass with one or more organic solvents at a temperature above about 100° C.

In some embodiments of any of the above methods, the contacting is performed at a pH of about 14.0, about 13.5, about 13.0, about 12.5, about 12.0, about 11.5, about 11.0, about 10.5, about 10.0, about 9.5, about 9.0, about 8.5, about 8.0, about 7.5, about 7.0, about 6.5, about 6.0, about 5.5, about 5.0, about 4.5, about 4.0, about 3.5, about 3.0, about 2.5, about 2.0, about 1.5, about 1.0, about 0.5, or about 0. In other embodiments of any of the above methods, the oligomers are desorbed from the MCM with an ionic liquid, an acid, an alcohol, water, a mixture of an alcohol and water, or a mixture of LiCl and N,N-dimethylacetamide. For example, the alcohol may be ethanol. In one embodiment, the mixture of ethanol and water contains a 60:40 volume/volume ratio of ethanol to water.

In other embodiments of any of the above methods, the MCM is a mesoporous carbon nanoparticle. In certain embodiments, the mesoporous carbon nanoparticle is selected from a CMK-1 type nanoparticle, a CMK-3 type nanoparticle, a CMK-5 type nanoparticle, and a CMK-8 type nanoparticle. In other embodiments, the mesoporous carbon nanoparticle has an average particle size that ranges from about 10 nm to about 200 nm.

In still other embodiments of any of the above methods, the MCM has a surface area that ranges from about 1000 m²/g to about 2500 m²/g. In yet other embodiments of any of the above methods, the MCM has a pore volume that ranges from about 1 cm³/g to about 2 cm³/g. In further embodiments of any of the above methods, the MCM has an average pore diameter that ranges from about 2.5 nm to about 5.0 nm.

In some embodiments of any of the above methods, the MCM further contains an acid-functionalized surface, a base-functionalized surface, or an acid/base-functionalized surface. In certain embodiments, the MCM containing an acid-functionalized surface further depolymerizes the adsorbed oligomers into shorter chain oligomers. In certain embodiments, the shorter chain oligomers are desorbed from the MCM with an ionic liquid, an acid, an alcohol, water, a mixture of an alcohol and water, or a mixture of LiCl and N,N-dimethylacetamide. For example, the alcohol may be ethanol. In other embodiments, the shorter chain oligomers are monosaccharides. In some embodiments, the monosaccharides are selected from glucose, xylose, and a combination thereof. In still other embodiments, from at least 50% to at least 95% of the oligomers are depolymerized to glucose. In certain embodiments, the monosaccharides are desorbed from the MCM with an ionic liquid, an acid, an alcohol, water, a mixture of an alcohol and water, or a mixture of LiCl and N,N-dimethylacetamide. In further embodiments, the acid is a mineral acid. Preferably, the mineral acid is a sulfonic acid or a phosphonic acid. In other embodiments, the acid is an organic acid. Preferably, the organic acid is a carboxylic acid.

In other embodiments of any of the above methods, the adsorption of the oligomers to the MCM prevents further depolymerization of the oligomers.

Other aspects of the present disclosure relate to a method of purifying oligomers from depolymerized biomass, by: a) providing biomass; b) depolymerizing the biomass to produce an oligomer-containing solution; c) providing a carbonaceous material; and d) contacting the oligomer-containing solution with the carbonaceous material under conditions whereby the MCM adsorbs at least one oligomer from the solution to purify the oligomers from the depolymerized biomass, where the contacting is performed at a pH of about 4.0 or below. In some embodiments, the method further includes desorbing the adsorbed oligomers from the carbonaceous material.

In some embodiments, the biomass is lignocellulosic biomass. In other embodiments, the biomass contains cellulose. In still other embodiments, the biomass contains hemicellulose. In further embodiments, the biomass is cellulosic biomass.

In some embodiments of any of the above methods, the oligomer-containing solution contains lignin oligomers. In other embodiments of any of the above methods, the oligomer-containing solution contains hemicellulose oligomers. Preferably, the hemicellulose oligomers are oligosaccharides. In certain embodiments, the oligosaccharides are selected from xylose, xylans, xyloglucans, mannans, mannose, galactose, rhamnose, arabinose, arabinoxylans, and combinations thereof.

In yet other embodiments of any of the above methods, the oligomer-containing solution contains poly-β-glucan fragments derived from cellulose. Preferably, the poly-β-glucan fragments contain oligosaccharides. In certain embodiments, the oligosaccharides are selected from glucose, cellobiose, cellotriose, cellotetraose, cellopentaose, cellohexaose, and combinations thereof.

In further embodiments of any of the above methods, the oligomer-containing solution contains long chain poly-β-glucans.

In some embodiments of any of the above methods, the depolymerizing includes contacting the biomass with water at a temperature above at least 50° C.

In other embodiments of any of the above methods, the depolymerizing includes acid hydrolysis. In certain embodiments, the depolymerizing includes contacting the solution with an acid. In some embodiments, the acid is a dilute acid. In other embodiments, the acid is a concentrated acid. In still other embodiment, the acid is a mineral acid. Preferably, the mineral acid is HCl, H₃PO₃, H₃PO₄, H₂SO₄, H₂SO₃, HF, HlO₄, HBr, H₃O⁺, HNO₂, HNO₃, HI, boronic acid, or polyoxometallate acid. In yet other embodiments, the acid is an organic acid. Preferably, the organic acid is acetic acid or carboxylic acid.

In other embodiments of any of the above methods, the depolymerizing includes contacting the biomass with hot water, one or more organic acids, one or more supercritical fluids, one or more near-supercritical fluids, or combinations thereof. In still other embodiments of any of the above methods, the depolymerizing includes contacting the biomass with one or more ionic liquids. In certain embodiments, the contacting occurs at a temperature above about 50° C. In yet other embodiments of any of the above methods, the depolymerizing includes contacting the biomass with one or more organic solvents at a temperature above about 100° C.

In some embodiments of any of the above methods, the contacting is performed at a pH of about 3.5 or below, about 3.0 or below, about 2.5 or below, about 2.0 or below, about 1.5 or below, or about 1.0 or below. In other embodiments of any of the above methods, the oligomers are desorbed from the carbonaceous material with an ionic liquid, an acid, an alcohol, water, a mixture of an alcohol and water, or a mixture of LiCl and N,N-dimethylacetamide. For example, the alcohol may be ethanol. In one embodiment, the mixture of ethanol and water contains a 60:40 volume/volume ratio of ethanol to water.

In certain embodiments of any of the above methods, the carbonaceous material is selected from activated charcoal, activated coal, activated carbon, powdered activated carbon, granular activated carbon, extruded activated carbon, bead activated carbon, impregnated carbon, polymer coated carbon, and mesoporous carbon material. In other embodiments of any of the above methods, the carbonaceous material is activated charcoal.

In other embodiments of any of the above methods, the carbonaceous material is mesoporous carbon material. In certain preferred embodiments, the mesoporous carbon material is a mesoporous carbon nanoparticle. In some embodiments, the mesoporous carbon nanoparticle is selected from a CMK-1 type nanoparticle, a CMK-3 type nanoparticle, a CMK-5 type nanoparticle, and a CMK-8 type nanoparticle. In other embodiments, the mesoporous carbon nanoparticle has an average particle size that ranges from about 10 nm to about 200 nm.

In still other embodiments of any of the above methods, the carbonaceous material has a surface area that ranges from about 1000 m²/g to about 2500 m²/g. In yet other embodiments of any of the above methods, the carbonaceous material has a pore volume that ranges from about 1 cm³/g to about 2 cm³/g. In further embodiments of any of the above methods, the carbonaceous material has an average pore diameter that ranges from about 2.5 nm to about 5.0 nm.

In some embodiments of any of the above methods, the carbonaceous material further contains an acid-functionalized surface, a base-functionalized surface, or an acid/base-functionalized surface. In certain embodiments, the carbonaceous material containing an acid-functionalized surface further depolymerizes the adsorbed oligomers into shorter chain oligomers. In certain embodiments, the shorter chain oligomers are desorbed from the MCM with an ionic liquid, an acid, an alcohol, water, a mixture of an alcohol and water, or a mixture of LiCl and N,N-dimethylacetamide. In other embodiments, the shorter chain oligomers are monosaccharides. For example, the alcohol may be ethanol. In some embodiments, the monosaccharides are selected from glucose, xylose, and a combination thereof. In still other embodiments, from at least 50% to at least 95% of the oligomers are depolymerized to glucose. In certain embodiments, the monosaccharides are desorbed from the MCM with an ionic liquid, an acid, an alcohol, water, a mixture of an alcohol and water, or a mixture of LiCl and N,N-dimethylacetamide. In further embodiments, the acid is a mineral acid. Preferably, the mineral acid is a sulfonic acid or a phosphonic acid. In other embodiments, the acid is an organic acid. Preferably, the organic acid is a carboxylic acid.

In other embodiments of any of the above methods, the adsorption of the oligomers to the carbonaceous material prevents further depolymerization of the oligomers.

In other aspects, provided is a method of isolating at least one monosaccharide from an oligomer-adsorbed carbonaceous material, by: a) providing an oligomer-adsorbed carbonaceous material, where the oligomers adsorbed on the carbonaceous material are polysaccharides; b) contacting the oligomer-adsorbed carbonaceous material with an acid, water, or a combination thereof to hydrolyze at least a portion of the polysaccharides into monosaccharides; and c) isolating at least one of the monosaccharides produced in step (b). In some embodiments, the carbonaceous material is a mesoporous carbon material (MCM).

In some embodiments, the acid is a dilute acid. In other embodiments, the acid is a concentrated acid. In still other embodiment, the acid is a mineral acid. Preferably, the mineral acid is HCl, H₃PO₃, H₃PO₄, H₂SO₄, H₂SO₃, HF, HlO₄, HBr, H₃O⁺, HNO₂, HNO₃, HI, boronic acid, or polyoxometallate acid. In yet other embodiments, the acid is an organic acid. Preferably, the organic acid is acetic acid or carboxylic acid. In some embodiment, the water is hot water. In certain embodiments, the method may be performed at temperatures of at least 90° C., for example, at 100° C. to 150° C., or at 125° C.

In certain embodiments, the providing of the oligomer-adsorbed carbonaceous material includes: providing biomass; depolymerizing the biomass to produce an oligomer-containing solution, where the oligomer-containing solution includes polysaccharides; providing a carbonaceous material; and contacting the oligomer-containing solution with the carbonaceous material under conditions where the carbonaceous material adsorbs at least one polysaccharide from the solution. In one embodiment, the polysaccharides are selected from glucan, xylan, and a combination thereof. In another embodiment, the monosaccharides are selected from glucose, xylose, and a combination thereof.

In yet other aspects, provided is a method of separating a mixture of oligomers, by: a) providing a mixture of oligomers, where the mixture includes C5 oligomers and C6 oligomers; b) providing a mesoporous carbon material (MCM); and c) contacting the MCM with the mixture of oligomers under conditions whereby the MCM adsorbs at least one C6 oligomer to separate the mixture of the oligomers. In some embodiments, the method further includes desorbing at least one adsorbed C6 oligomer. In one embodiment, the C5 oligomers are selected from xylose, arabinose, and a combination thereof. In another embodiment, the C6 oligomers are selected from glucose, glucan, cello-oligosaccharides, and a combination thereof.

Further aspects of the present disclosure relate to an acid-functionalized, a base-functionalized, or an acid/base-functionalized mesoporous carbon material (MCM). In one embodiment, the MCM is acid-functionalized, containing a phosphonic acid or a carboxylic acid. In another embodiment, the MCM is base-functionalized, containing phenolate or carboxylate. In yet another embodiment, the MCM is acid/base-functionalized, containing phenolic acid and phenolate as the acid and the base, respectively. In yet another embodiment, the MCM is acid/base-functionalized, containing carboxylic acid and carboxylate as the acid and the base, respectively.

In some embodiments, the MCM is a mesoporous carbon nanoparticle. In other embodiments, the mesoporous carbon nanoparticle is selected from a CMK-1 type nanoparticle, a CMK-3 type nanoparticle, a CMK-5 type nanoparticle, and a CMK-8 type nanoparticle. In yet other embodiments, the mesoporous carbon nanoparticle has an average particle size that ranges from about 10 nm to about 200 nm.

In other embodiments of any of the above MCM, the MCM has a surface area that ranges from about 1000 m²/g to about 2500 m²/g. In still other embodiments of any of the above MCM, the MCM has a pore volume that ranges from about 1 cm3/g to about 2 cm3/g. In further embodiments of any of the above MCM, the MCM has an average pore diameter that ranges from about 2.5 nm to about 5.0 nm.

In some embodiments of any of the above MCM, the MCM adsorbs lignocellulosic oligomers. In certain embodiments, the adsorption capacity of the MCM for the oligomers ranges from at least 40% to at least 95% of oligomer mass uptake relative to the mass of the MCM. In other embodiments, he oligomers are selected from lignin oligomers, hemicellulose oligomers, poly-β-glucan fragments derived from cellulose, oligosaccharides, long chain poly-β-glucans, glucose, cellobiose, cellotriose, cellotetraose, cellopentaose, cellohexaose, xylose, xylans, xyloglucans, mannans, mannose, galactose, rhamnose, arabinose, arabinoxylans, and combinations thereof. In still other embodiments, the MCM adsorbs oligomers at a pH of about 7.0, about 6.5, about 6.0, about 5.5, about 5.0, about 4.5, about 4.0, about 3.5, about 3.0, about 2.5, about 2.0, about 1.5, about 1.0, about 0.5, or about 0. In yet other embodiments, from at least 50% to at least 95% of the oligomers are depolymerized to glucose. In further embodiments, the oligomers are desorbed from the MCM with an ionic liquid, an acid, an alcohol, water, a mixture of an alcohol and water, or a mixture of LiCl and N,N-dimethylacetamide. For example, the alcohol may be ethanol. In one embodiment, the mixture of ethanol and water contains a 60:40 volume/volume ratio of ethanol to water.

### BRIEF DESCRIPTION OF THE DRAWINGS

FIG. 1 schematically shows a procedure for synthesizing mesoporous carbon nanoparticles.

FIG. 2 graphically depicts adsorption isotherms of glucose on mesoporous carbon nanoparticles.

FIG. 3 graphically depicts adsorption isotherms of cellobiose on mesoporous carbon nanoparticles.

FIG. 4A depicts the adsorption of oligosaccharides on mesoporous carbon nanoparticles. FIG. 4B depicts the desorption of the oligosaccharides from mesoporous carbon nanoparticles.

FIG. 5 schematically shows a procedure for synthesizing an acid-treated cellulose solution.

FIG. 6 depicts the results of HPLC showing the adsorption of poly-β-glucans derived from cellulose on mesoporous carbon nanoparticles.

FIG. 7 depicts HPLC analysis of cellulose solution after acid hydrolysis.

FIG. 8 depicts MALDI-TOF-MS spectra of adsorbed small chain and long chain oligosaccharides on mesoporous carbon nanoparticles.

FIG. 9 schematically shows a procedure for synthesizing sulfonated mesoporous carbon nanoparticles.

FIG. 10 graphically depicts the hydrolysis of cellobiose by sulfonated mesoporous carbon nanoparticles.

FIG. 11 graphically depicts the conversion of cellobiose to glucose by sulfonated mesoporous carbon nanoparticles.

FIG. 12 schematically shows the hydrolysis of cellulose to glucose by sulfonated mesoporous carbon nanoparticles.

FIG. 13 schematically shows a method of synthesizing phosphonate-functionalized mesoporous carbon material.

FIG. 14 depicts the coverage of the phosphonic acid functionality of phosphonic acid-functionalized mesoporous material.

FIG. 15 depicts an exemplary process for separating a mixture of C5 and C6 sugars using a MCN.

FIG. 16 shows three ¹³C DP-MAS solid-state nuclear magnetic resonance spectra of: (a) ¹³C-labeled crystalline cellulose prior to use in Example 14; (b) ¹³C-labeled poly β-glucan adsorbed on the MCN at a lower concentration; and (c) ¹³C-labeled poly β-glucan adsorbed on the MCN at a higher concentration.

FIG. 17 is a graph showing oligosaccharide/glucan distribution following desorption from the MCN using 0.5% wt LiCl/DMAc solvent at room temperature. The solid line ( [symbol] ) represents an adsorbed concentration of 38 mg·g⁻¹ on the MCN, whereas the dashed line ( [symbol] ) represents an adsorbed concentration fraction of 303 mg·g⁻¹ on the MCN.

FIG. 18 is an exemplary process of glucan adsorption onto MCN.

FIG. 19 is an exemplary process flow diagram for hydrolysis of xylan adsorbed carbon materials.

### DETAILED DESCRIPTION

Overview

The present disclosure relates to methods of purifying oligomers, by: a) providing an oligomer-containing solution that may be obtained from depolymerizing biomass; b) providing a carbonaceous material; c) contacting the oligomer-containing solution with the carbonaceous material under conditions whereby the carbonaceous material adsorbs at least one oligomer from the solution; and d) desorbing the adsorbed oligomers from the carbonaceous material to purify the at least one oligomer from the depolymerized biomass.

The carbonaceous material may be, for example, a mesoporous carbon material (MCM). In one embodiment, the carbonaceous material is a mesoporous carbon nanoparticle (MCN). The surface of the carbonaceous material may also be functionalized. In certain embodiments, the carbonaceous material is acid-functionalized, base-functionalized, or acid/base-functionalized. For example, in one embodiment, the carbonaceous material is an acid-functionalized mesoporous carbon material (MCM), containing a phosphonic acid or a carboxylic acid. The phosphonic acid may be, for example, phosphoric acid. The carboxylic acid may be, for example, acetic acid. In another embodiment, the carbonaceous material is a base-functionalized mesoporous carbon material (MCM), containing phenolate or carboxylate. In yet another embodiment, the carbonaceous material is acid/base-functionalized mesoporous carbon material (MCM), containing phenolic acid and phenolate as the acid/base pair, or carboxylic acid and carboxylate as the acid/base pair.

The present disclosure is based, at least in part, on the novel discovery that a MCM having a surface area of about 2000 m²/g and an average pore diameter of about 2.4 nm were able to adsorb cellulosic oligosaccharides with glucose chain lengths ranging in size from 1 to at least 6 glucose molecules. Advantageously, the MCM was able to adsorb oligosaccharides at a low pH and with a high adsorption capacity of about 50% of oligosaccharide mass uptake relative to the mass of the mesoporous carbon. Additionally, it was found that when this MCM was sulfonated, the acid-functionalized MCM was able to adsorb cellulose and hydrolyze it to oligosaccharides. Advantageously, when cellobiose was treated with the sulfonated MCM, approximately 87% of the cellobiose was hydrolyzed to glucose.

### DEFINITIONS

Unless defined otherwise, all scientific and technical terms are understood to have the same meaning as commonly used in the art to which they pertain. For the purpose of the present disclosure, the following terms are defined.

As used herein, “carbonaceous material(s)” refers to a carbon-based adsorbent material.

As used herein, “mesoporous” refers to a porous material having pores with a diameter that ranges from about 2 nm to about 50 nm.

As used herein, “mesoporous carbon material(s)” and “MCM” are used interchangeably and refer to a carbon-based mesoporous material.

As used herein, “nanoparticle” refers to a particle having a particle size that is less than about 250 nm and that behaves as a whole unit in terms of its transport and properties.

As used herein, “lignocellulose” refers to any material primarily consisting of cellulose, hemicellulose, and lignin.

As used herein, “cellulose” refers to a polysaccharide containing a linear chain of several hundred to several thousand β(1→4) linked D-glucose monosaccharides.

As used herein, “hemicellulose” refers to a polymer of short, highly-branched chains of mostly five-carbon pentose sugars (e.g., xylose and arabinose) and to a lesser extent six-carbon hexose sugars (e.g., galactose, glucose and mannose).

As used herein, “oligosaccharides” refers to monosaccharides, disaccharides, and saccharide polymers containing from three to fifteen component monosaccharides. For example, cellohexaose contains six glucose monomers, cellopentaose contains five glucose monomers, cellotetraose contains four glucose monomers, cellotriose contains three glucose monomers, and cellobiose contains two glucose monomers.

As used herein, an “oligomer-containing solution” refers to a solution that contains fragments of lignin, hemicellulose, and cellulose that are produced by the hydrolysis of lignocellulosic biomass.

As used herein, “hemicellulose oligomers” refers to hemicellulose fragments produced by the hydrolysis of lignocellulosic biomass.

As used herein, “lignin oligomers” refers to lignin fragments produced by the hydrolysis of lignocellulosic biomass.

As used herein, “poly-β-glucan fragments derived from cellulose” refers to poly-β-glucan fragments produced by the hydrolysis of the cellulose fraction of lignocellulosic biomass.

As used herein, “depolymerization” refers to the break-down or decomposition of long chain polysaccharide chains to oligosaccharides. One non-limiting example of a type depolymerization is hydrolysis.

As used herein, “hydrolysis”, “hydrolyzing”, and “saccharification” are used interchangeably and refer to the chemical or enzymatic process of cleaving glycosidic bonds on polysaccharides, and oligosaccharides to yield shorter length oligosaccharides.

As used herein, “purifying oligomers” refers to concentrating the amount of oligomers from a mixture or solution. For example, oligomers may be purified from an oligomer-containing solution or depolymerized biomass by removing at least one oligomer from the solution or mixture. In some embodiments, the methods provided herein involve purifying oligomers by removing oligomers from a solution or mixture by adsorption on a carbonaceous material, such as a MCM. In other embodiments, the methods provided herein involve purifying oligomers by separating two or more types of oligomers (e.g., C6 oligomers from C5 oligomers) using the carbonaceous material, such as MCM. It should be understood that oligomers may include oligosaccharides. It should also be understood that the depolymerized biomass may include a mixture of oligosaccharides, insoluble biomass debris, non-depolymerized material, lignin, and any combinations thereof.

### Depolymerization of Biomass

Certain aspects of the present disclosure relate to methods of purifying oligomers produced by depolymerizing biomass for use, e.g., in the production of biofuels. As disclosed herein “biomass” refers to mass obtained from living matter, such as plants, algae, fungi, bacteria, and bacterial biofilms. Biomass of the present disclosure may include lignocellulosic biomass and/or cellulosic biomass.

In some embodiments, biomass of the present disclosure is biomass obtained from plant biomass. Suitable plant biomass includes, without limitation, Miscanthus, energy grass, elephant grass, switchgrass, cord grass, rye grass, reed canary grass, common reed, wheat straw, barley straw, canola straw, oat straw, corn stover, soybean stover, oat hulls, oat spelt, sorghum, rice hulls, sugarcane bagasse, corn fiber, barley, oats, flax, wheat, linseed, citrus pulp, cottonseed, groundnut, rapeseed, sunflower, peas, lupines, palm kernel, coconut, konjac, locust bean gum, gum guar, soy beans, Distillers Dried Grains with Solubles (DDGS), Blue Stem, corncobs, pine, conifer softwood, eucalyptus, birchwood, willow, aspen, poplar wood, hybrid poplar, energy cane, short-rotation woody crop, crop residue, yard waste, or a combination thereof.

In certain embodiments, the biomass contains lignocellulosic biomass. As disclosed herein, the lignocellulosic biomass may contain cellulose and/or hemicellulose.

In other embodiments, biomass of the present disclosure is cellulosic biomass obtained from algae, fungi, bacteria, and bacterial biofilms.

In certain embodiments, the oligomer-containing solution produced from the depolymerization of biomass contains lignin oligomers.

In other embodiments, the oligomer-containing solution produced from the depolymerization of biomass contains hemicellulose oligomers. In some embodiments the hemicellulose oligomers are oligosaccharides. Oligosaccharides derived from hemicellulose include, without limitation, xylose, xylans, xyloglucans, mannans, mannose, galactose, rhamnose, arabinose, arabinoxylans, and combinations thereof.

In further embodiments, the oligomer-containing solution produced from the depolymerization of biomass contains poly-β-glucan fragments derived from cellulose. In some embodiments, the poly-β-glucan fragments contain oligosaccharides. Examples of oligosaccharides derived from poly-β-glucan include, without limitation, glucose, cellobiose, cellotriose, cellotetraose, cellopentaose, cellohexaose, and combinations thereof.

In other embodiments, the oligomer-containing solution produced from the depolymerization of biomass contains long chain poly-β-glucans.

In certain embodiments, depolymerizing biomass includes pretreating biomass. Biomass that is used as a feedstock, for example, in biofuel production, generally contains high levels of lignin, which can block depolymerization of the cellulosic and/or hemicellulosic components of the biomass. Typically, the biomass is pretreated to increase the accessibility of the components to depolymerization. Methods of pretreating lignocellulosic biomass are well known in the art and include, without limitation, steam explosion, ammonia fiber expansion (AFEX), CO₂ explosion, ozone pre-treatment, and mechanical pretreatment.

In other embodiments, depolymerizing biomass also includes contacting the biomass with water (including, for example, hot water), one or more bases, one or more dilute acids, one or more organic acids, one or more organic solvents, one or more ionic liquids, one or more supercritical fluids, or one or more near-supercritical fluids. In some embodiments, the contacting occurs at high temperature and/or high pressure. In certain preferred embodiments, the contacting occurs at a temperature above about −20° C., above about −10° C., above about 0° C., above about 10° C., above about 20° C., above about 30° C., above about 35° C., above about 40° C., above about 45° C., above about 50° C., above about 55° C., above about 60° C., above about 65° C., above about 70° C., above about 75° C., above about 80° C., above about 85° C., above about 90° C., above about 95° C., above about 100° C., above about 110° C., above about 120° C., above about 130° C., above about 140° C., above about 150° C., above about 160° C., above about 170° C., above about 180° C., above about 190° C., above about 200° C., above about 300° C., above about 400° C., above about 500° C., above about 600° C., or higher. In other embodiments, the contacting occurs at a temperature between −20° C. and 100° C., or between 4° C. and 160° C. It should be understood that the temperature selected for this step may be suitable for adsorption, as preparation of the hydrolysate. It should also be noted that the temperatures described herein may vary by ±2° C. For example temperature of about 50° C. could vary from 48° C. to 52° C. In certain preferred embodiments, the biomass is contacted with water (including, for example, hot water), one or more organic acids, one or more supercritical fluids, one or more near-supercritical fluids, or combinations thereof. In other preferred embodiments, the biomass is contacted with one or more ionic liquids. In further preferred embodiments, the biomass is contacted with one or more organic solvents at a temperature above about 100° C.

In some embodiments, depolymerizing biomass includes, without limitation, acid hydrolysis, alkaline hydrolysis, and enzymatic hydrolysis.

Acid Hydrolysis

Examples of acid hydrolysis include, without limitation, those of U.S. Pat. Nos. 5,726,046 and 5,972,118. The acid hydrolysis may be either dilute acid hydrolysis or concentrated acid hydrolysis. Generally, acid hydrolysis involves treating biomass with an acid. The acid may be either dilute acid or concentrated acid. In some embodiments, the biomass is treated with acid at atmospheric pressure. In other embodiments, the biomass is treated with acid at greater than atmospheric pressure. In still other embodiments, the biomass is treated with acid at room temperature. In other embodiments, the biomass is treated with acid at a temperature greater than 40° C. In further embodiments, the biomass is treated with acid for at least 1 hr, at least 2 hr, at least 3 hr, at least 4 hr, at least 5 hr, at least 6 hr, at least 7 hr, at least 8 hr, at least 9 hr, at least 10 hr, at least 12 hr, at least 18 hr, at least 24 hr, at least 30 hr, at least 36 hr, at least 42 hr, at least 48 hr, at least 60 hr, at least 72 hr, at least 96 hr, or more.

In other embodiments, the acid hydrolysis is carried out at a pH of about 6.5, about 6.0, about 5.5, about 5.0, about 4.5, about 4.0, about 3.5, about 3.0, about 2.5, about 2.0, about 1.5, about 1.0, about 0.5, or about 0. It should be noted that the pH values described herein may vary by ±0.2. For example a pH value of about 6 could vary from pH 5.8 to pH 6.2.

In some embodiments, the acid is a mineral acid. As used herein “mineral acid” and “inorganic acid” are used interchangeably and refer to an acid derived from one or more inorganic compounds. Suitable inorganic acids include without limitation, hydrochloric acid (HCl), phosphorous acid (H₃PO₃), phosphoric acid (H₃PO₄), sulfuric acid (H₂SO₄), sulfurous acid (H₂SO₃), hydrofluoric acid (HF), perchloric acid (HlO₄), hydrobromic acid (HBr), hydronium (H₃O⁺), nitrous acid (HNO₂), nitric acid (HNO₃), hydroiodic acid (HI), boronic acid, and polyoxometallate acid. In other embodiments, the acid is an organic acid. For example, the organic acid may be acetic acid or a carboxylic acid.

Alkaline Hydrolysis

Generally, alkaline hydrolysis involves treating biomass with a base. In some embodiments, the biomass is treated with base at atmospheric pressure. In other embodiments, the biomass is treated with base at greater than atmospheric pressure. In still other embodiments, the biomass is treated with base at room temperature. In other embodiments, the biomass is treated with base at a temperature greater than 40° C. In further embodiments, the biomass is treated with base for at least 1 hr, at least 2 hr, at least 3 hr, at least 4 hr, at least 5 hr, at least 6 hr, at least 7 hr, at least 8 hr, at least 9 hr, at least 10 hr, at least 12 hr, at least 18 hr, at least 24 hr, at least 30 hr, at least 36 hr, at least 42 hr, at least 48 hr, at least 60 hr, at least 72 hr, at least 96 hr, or more.

In other embodiments, the alkaline hydrolysis is carried out at a pH of about 14.0, about 13.5, about 13.0, about 12.5, about 12.0, about 11.5, about 11.0, about 10.5, about 10.0, about 9.5, about 9.0, about 8.5, about 8.0, or about 7.5.

Bases suitable for alkaline hydrolysis may be dilute bases or concentrated bases. Examples of suitable bases include, without limitation, ammonia (NH₃), ammonium hydroxide (NH₄OH), sodium hydroxide (NaOH), potassium hydroxide (KOH), lithium hydroxide (LiOH), calcium hydroxide [Ca(OH)₂], magnesium hydroxide [Mg(OH)₂], sodium carbonate (Na₂CO₃), potassium carbonate (K₂CO₃), and calcium carbonate (CaCO₃). In certain preferred embodiments, the base is dilute ammonia, NH₄OH, NAOH, KOH, or LiOH.

Enzymatic Hydrolysis

As used herein, “enzymatic hydrolysis” refers to the hydrolytic process of biomass, such as lignocellulosic or cellulosic biomass, by one or more enzymes or cellulases to produce oligosaccharides. Hydrolysis enzymes catalyze the conversion of biomass into oligosaccharides. Hydrolysis enzymes are well known in the art and include, without limitation, cellulases, endoglucanases, exoglucanases, hemicellulases, β-glucosidases, xylanases, endoxylanases, β-xylosidases, arabinofuranosidases, glucuronidases, and acetyl xylan esterases. Combinations of enzymes (i.e., enzyme cocktails) can also be tailored to the structure of a specific lignocellulosic and/or cellulosic biomass feedstock to increase the level of degradation. In certain embodiments, commercial cellulase mixtures may be used. Examples of commercially available cellulase mixtures include, without limitation, Celluclast 1.5 L® (Novozymes), Spezyme CP® (Genencor), and Cellulyve 50 L® (Lyven).

In certain embodiments, biomass of the present disclosure is hydrolyzed under suitable conditions to produce oligosaccharides. Much is known about factors that relate to enzymatic hydrolysis. Hydrolysis rates increase with temperature, but at too high a temperature the enzymes will become denatured. High solids are desirable for high titer, but the percentage of theoretical hydrolysis achieved decreases with increased solids. It has been hypothesized that this was due to inhibition by the products of hydrolysis (see, Kristensen, et al., Biotechnology for Biofuels (2009) 2, 11). This effect is strong enough to make 20% solids a practical upper limit for enzymatic hydrolysis. Moreover, the addition of enzymes above 20% solids in an integrated process is not expected to have the same level of hydrolytic performance as a process at a lower consistency, such as 15%.

The methods and conditions suitable for enzymatic hydrolysis to convert biomass into oligosaccharides are well known in the art. For example, Tengborg et al. teach one way for enzymatic hydrolysis of steam-pretreated softwood, such as spruce, for sugar production (see Tengborg et al., Biotechnol. Prog. (2001) 17: 110-117).

In some embodiments, one or more hydrolysis enzymes are added to the biomass and incubated under suitable conditions for the enzymes to hydrolyze the biomass.

In other embodiments, microorganisms expressing one or more hydrolysis enzymes are contacted with the biomass and cultured under suitable conditions for the expressed one or more hydrolysis enzymes to hydrolyze the biomass. Suitable microorganisms include, without limitation, fungi, yeast, bacteria, and algae. In some embodiments, the microorganisms endogenously express the one or more hydrolysis enzymes. In other embodiments, the microorganisms recombinantly express the one or more hydrolysis enzymes.

### Carbonaceous Materials

Other aspects of the present disclosure relate to methods of purifying oligomers from depolymerized biomass by utilizing carbonaceous material, such as mesoporous carbon materials, to adsorb the oligomers produced from the depolymerization of biomass.

Carbonaceous material suitable for the methods and compositions of the present disclosure may be any carbonaceous materials known to one of ordinary skill in the art, and include, without limitation, activated charcoal, activated coal, activated carbon, powdered activated carbon, granular activated carbon, extruded activated carbon, bead activated carbon, impregnated carbon, and polymer coated carbon. Methods of producing carbonaceous material are well known in the art and include, without limitation, carbonization by pyrolizing carbon material with gases (e.g., argon or nitrogen) at high temperatures, activation/oxidation by exposing carbon material to oxidizing gases (e.g., carbon dioxide, oxygen, or steam) at high temperatures, and chemical activation by impregnating carbon material with chemicals such as acids, strong bases, or ionic salts at high temperatures. Carbon sources that may be utilized to produce carbonaceous material include, without limitation coal and charcoal.

In certain preferred embodiments, carbonaceous material of the present disclosure is utilized in any of the disclosed methods at a pH of about 4.0 or below, 3.5 or below, about 3.0 or below, about 2.5 or below, about 2.0 or below, about 1.5 or below, or about 1.0 or below.

In some embodiments, carbonaceous material of the present disclosure is mesoporous carbon material. Mesoporous carbon materials (MCM) of the present disclosure include, without limitation, any carbon-based materials known to one of ordinary skill in the art that are porous with a pore diameter that ranges from about 2 nm to about 50 nm. Examples of such MCM include, without limitation, activated mesoporous carbon, amorphous mesoporous carbon materials, structurally ordered mesoporous carbon materials, mesoporous carbon nanofibers, mesoporous carbon nanotubes, and mesoporous carbon nanoparticles. Methods of producing such MCM include, without limitation, nanocasting, evaporation induced self-assembly (EISA), chemical vapor deposition (CVD), hard-template synthesis, silica particle-template synthesis, colloidal silica particle-template synthesis, silica/aluminosilicate gel-template synthesis, anodic aluminum-template synthesis, polymer bead-template synthesis, soft-template synthesis, micelle-template synthesis, copolymerization/cocondensation synthesis, and synthesis by self-assembly of copolymers (see, Jun et al., J. Am. Chem. Soc. 2000, 122, 10712-10713; Kim et al., Nano Letters, 2008, 8. 11: 3724-3727; Liang et al., Angew. Chem. Int. Ed. 2008, 47, 3696-3717; and Fang et al., Angew. Chem. Int. Ed. 2010, 49, 7987-7991).

Carbon sources that may be utilized to synthesize MCM include, without limitation, furfuryl alcohol, cellulose, carbides, phenol resins, saccharides, and sucrose. Silica templates that may be utilized to synthesize MCM include, without limitation, the ordered aluminosilicate MCM-48, MCM-41 silica, the hexagonal mesoporous aluminosilicate Al-HMS, and the hexagonally structured silica SBA-15.

As disclosed herein, the average particle size, surface area, pore volume, and average pore diameter of carbonaceous material of the present disclosure, such as MCM, are generally determined by the method of synthesis, the template used, and the carbon material used for synthesizing the carbonaceous material.

In certain embodiments, the carbonaceous material, such as MCM, is a mesoporous carbon nanoparticle. Examples of mesoporous carbon nanoparticles include, without limitation, CMK-1 type nanoparticles, CMK-3 type nanoparticles, CMK-4 type nanoparticles, CMK-5 type nanoparticles, CMK-8 type nanoparticles, and SNU-1 type nanoparticles. In certain preferred embodiments, the mesoporous carbon nanoparticles are CMK-1, CMK-3, CMK-5, or CMK-8 type nanoparticles.

Mesoporous carbon nanoparticles of the present disclosure may be synthesized by utilizing a silica particle-template. Silica particle-template synthesis generally involves polymerizing carbon material around the silica particle and then dissolving the silica template with an acid. The silica template can be dissolved with acids such as hydrofluoric acid.

In some embodiments, mesoporous carbon nanoparticles of the present disclosure have an average particle size that is about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 225 nm, about 250 nm, about 275 nm, or about 300 nm. In other embodiments, the average particle size ranges from about 10 nm to about 300 nm. In certain preferred embodiments, the average particle size ranges from about 10 nm to about 200 nm. It should be noted that the average particle sizes described herein may vary by ±2 nm. For example an average particle size of about 100 nm could vary from 98 nm to 102 nm. Methods of determining the average particle size of mesoporous carbon nanoparticles are well known in the art and include transmission electron microscopy (TEM).

In other embodiments, carbonaceous material of the present disclosure, such as MCM, have a surface area of about 500 m²/g, 600 m²/g, 700 m²/g, 800 m²/g, 900 m²/g, 1000 m²/g, 1100 m²/g, 1200 m²/g, 1300 m²/g, 1400 m²/g, 1500 m²/g, 1600 m²/g, 1700 m²/g, 1800 m²/g, 1900 m²/g, 2000 m²/g, 2100 m²/g, 2200 m²/g, 2300 m²/g, 2400 m²/g, 2500 m²/g, 2600 m²/g, 2700 m²/g, 2800 m²/g, 2900 m²/g, or 3000 m²/g. In some embodiments, the surface area ranges from about 500 m²/g to about 2500 m²/g. In certain preferred embodiments, the surface area ranges from about 1000 m²/g to about 2500 m²/g. Preferably, the surface area is about 2000 m²/g. It should be noted that the surface areas described herein may vary by ±2 m²/g. For example a surface area of about 2000 m²/g could vary from 1998 m²/g to 2002 m²/g. Methods of calculating the surface area of carbonaceous material are well known in the art and include the multipotent Brunauer-Emett-Teller (BET) model from adsorption data.

In still other embodiments, carbonaceous material of the present disclosure, such as MCM, have a pore volume of about 0.1 cm³/g, about 0.2 cm³/g, about 0.3 cm³/g, about 0.4 cm³/g, about 0.5 cm³/g, about 0.6 cm³/g, about 0.7 cm³/g, about 0.8 cm³/g, about 0.9 cm³/g, about 1.0 cm³/g, about 1.1 cm³/g, about 1.2 cm³/g, about 1.3 cm³/g, about 1.4 cm³/g, about 1.5 cm³/g, about 1.6 cm³/g, about 1.7 cm³/g, about 1.8 cm³/g, about 1.9 cm³/g, about 2.0 cm³/g, about 2.1 cm³/g, about 2.2 cm³/g, about 2.3 cm³/g, about 2.4 cm³/g, about 2.5 cm³/g, about 2.6 cm³/g, about 2.7 cm³/g, about 2.8 cm³/g, about 2.9 cm³/g, or about 3.0 cm³/g. In some embodiments, the pore volume ranges from about 0.1 cm³/g to about 3.0 cm³/g. In certain preferred embodiments, the pore volume ranges from about 1.0 cm³/g to about 2.0 cm³/g. Preferably, the pore volume is about 1.0 cm³/g. It should be noted that the pore volumes described herein may vary by ±0.2 cm³/g. For example a pore volume of about 1.0 cm³/g could vary from 0.8 cm³/g to 1.2 cm³/g. Methods of calculating the pore volume of carbonaceous material are well known in the art and include the V-t plot method of calculating the pore volume.

In further embodiments, carbonaceous material of the present disclosure, such as MCM, have an average pore diameter of about 1.0 nm, about 1.2 nm, about 1.4 nm, about 1.6 nm, about 1.8 nm, about 2.0 nm, about 2.2 nm, about 2.4 nm, about 2.6 nm, about 2.8 nm, about 3.0 nm, about 3.2 nm, about 3.4 nm, about 3.6 nm, about 3.8 nm, about 4.0 nm, about 4.2 nm, about 4.4 nm, about 4.6 nm, about 4.8 nm, about 5.0 nm, about 5.2 nm, about 5.4 nm, about 5.6 nm, about 5.8 nm, or about 6.0 nm. In some embodiments, the average pore diameter ranges from about 1.0 nm to about 5.0 nm. In certain preferred embodiments, the average pore diameter ranges from about 2.0 nm to about 5.0 nm. Preferably, the average pore diameter is about 2.5 nm. It should be noted that the average pore diameters described herein may vary by ±0.2 nm. For example an average pore diameter of about 2.5 nm could vary from 2.3 nm to 2.7 nm. Methods of determining the average pore diameter of carbonaceous material are well known in the art and include nitrogen (N₂) adsorption/desorption.

Oligomer Adsorption

Other aspects of the present disclosure relate to contacting an oligomer-containing solution, such as an oligosaccharide solution, with carbonaceous material, such as mesoporous carbon materials (MCM), under conditions whereby the carbonaceous material adsorbs oligomers from the solution. Current methods of depolymerizing biomass, such as lignocellulosic biomass, generally waste approximately 15% of the resulting depolymerized biomass oligomers in solution. However, the methods of the present disclosure for purifying oligomers are able to preserve the approximately 15% of the resulting oligomers by utilizing carbonaceous material of the present disclosure, such as MCM, to sequester the oligomers.

The oligomer-containing solution may include polysaccharides, monosaccharides, and mixtures thereof. In certain embodiments, the oligomer-containing solution has a ratio of polysaccharides to monosaccharides, where the ratio is between 5 and 20, or between 9 and 12.

Conditions for adsorbing oligomers onto carbonaceous material, such as MCM, generally include incubating a oligomer-containing solution with MCM for at least 5 min, at least 10 min, at least 15 min, at least 25 min, at least 30 min, at least 45 min, at least 1 hr, at least 2 hr, at least 3 hr, at least 4 hr, at least 5 hr, at least 6 hr, at least 12 hr, at least 18 hr, 24 hr, at least 36 hr, at least 48 hr, at least 60 hr, at least 72 hr, at least 96 hr, or more and at a suitable pH temperature for the carbonaceous material to adsorb the oligomers.

In certain embodiments, the oligomer-containing solution is contacted with carbonaceous material, such as MCM, at a pH of about 14.0, about 13.5, about 13.0, about 12.5, about 12.0, about 11.5, about 11.0, about 10.5, about 10.0, about 9.5, about 9.0, about 8.5, about 8.0, about 7.5, about 7.0, about 6.5, about 6.0, about 5.5, about 5.0, about 4.5, about 4.0, about 3.5, about 3.0, about 2.5, about 2.0, about 1.5, about 1.0, about 0.5, or about 0. In embodiments where the oligomer-containing solution is produced by alkaline hydrolysis, the solution is contacted with carbonaceous material, such as MCM, at a pH of about 14.0, about 13.5, about 13.0, about 12.5, about 12.0, about 11.5, about 11.0, about 10.5, about 10.0, about 9.5, about 9.0, about 8.5, about 8.0, or about 7.5. In embodiments where the oligomer-containing solution is produced by acid hydrolysis, the solution is contacted with carbonaceous material, such as MCM, at a pH of about 6.5, about 6.0, about 5.5, about 5.0, about 4.5, about 4.0, about 3.5, about 3.0, about 2.5, about 2.0, about 1.5, about 1.0, about 0.5, or about 0. In certain preferred embodiment, the solution is contacted with carbonaceous material, such as MCM, at a pH of about 5.0, about 4.5, about 4.0, about 3.5, about 3.0, about 2.5, about 2.0, about 1.5, about 1.0, about 0.5, or about 0.

In other embodiments, the oligomer-containing solution is contacted with carbonaceous material, such as MCM, at a temperature of at least 15° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., at least 100° C., or higher. In certain preferred embodiments, the oligomer-containing solution is contacted with carbonaceous material, such as MCM, at a temperature that ranges from about 20° C. to about 30° C.

Carbonaceous material of the present disclosure, such as MCM, has a high adsorption capacity for biomass oligomers, such as lignocellulosic and/or cellulosic biomass oligomers. Methods for calculating the adsorption capacity of carbonaceous material, such as MCM, are well known in the art and include high-performance liquid chromatography (HPLC) analysis. In some embodiments, the adsorption capacity of carbonaceous material of the present disclosure, such as MCM, for oligomers is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more of oligomer mass uptake relative to the mass of the carbonaceous material. In other embodiments, the adsorption capacity of carbonaceous material, such as MCM, for oligomers ranges from at least 40% to at least 95% of oligomer mass uptake relative to the mass of the carbonaceous material.

In some embodiments, the adsorption capacity of carbonaceous material, such as MCM, for oligomers is at least 2 times, at least 3 times, at least 5 times, at least 7 times, at least 10 times, at least 15 times, at least 20 times, at least 25 times, at least 30 times, at least 35 times, at least 40 times, at least 45 times, at least 50 times, at least 55 times, at least 60 times, at least 70 times, at least 80 times, or at least 90 times by mass as compared to a carbon nanoparticles without internal mesoporosity.

In certain embodiments, the carbonaceous material of the present disclosure, such as MCM, adsorbs long-chained oligomers in a capacity of up to 30% by mass of the carbonaceous material in a way that preferentially adsorbs these long-chain oligomers. For example, long-chain oligomers may have at least 10 units, at least 20 units, at least 30 units, at least 40 units, at least 50 units, at least 60 units, or at least 70 units, or between 10 and 100 units, between 40 and 70 units, or have 40 units, 50 units, 60 units, or 70 units. The adsorption equilibrium time is unexpectedly fast, and in some embodiments, can be less than 10 minutes, 5 minutes, 4, minutes, 3 minutes, 2 minutes or 1 minute. This is in contrast to other carbonaceous materials that lack internal mesoporosity, such as graphite-type carbon nanopowders (CNP).

In other embodiments, the carbonaceous material of the present disclosure, such as MCM, adsorbs between 0 and 2000, between 20 and 700, or between 20 and 500 mg glucose equivalent per g of carbonaceous material.

Examples of oligomers that are adsorbed by carbonaceous material of the present disclosure, such as MCM, include, without limitation, lignin oligomers, hemicellulose oligomers, poly-β-glucan fragments derived from cellulose, oligosaccharides, long chain poly-β-glucans, glucose, cellobiose, cellotriose, cellotetraose, cellopentaose, cellohexaose, xylose, xylans, xyloglucans, mannans, mannose, galactose, rhamnose, arabinose, arabinoxylans, and any combinations thereof.

In certain embodiments, adsorbing biomass oligomers to carbonaceous material of the present disclosure, such as MCM, protects the oligomers from further depolymerization or hydrolysis.

In embodiments where depolymerizing biomass, such as lignocellulosic biomass, includes contacting the biomass with water (e.g., hot water), one or more bases, one or more dilute acids, one or more organic acids, one or more organic solvents one or more ionic liquids, one or more supercritical fluids, or one or more near-supercritical fluids, carbonaceous material of the present disclosure, such as MCM, may be used to purify oligomers, such as the lignin fraction or polysaccharide fraction, away from fractions of the biomass that will undergo a further depolymerization step to obtain short chain oligomers (e.g., oligosaccharides, disaccharides, and monosaccharides).

Oligomer Desorption

Once carbonaceous material of the present disclosure, such as MCM, has adsorbed biomass oligomers from solution, the carbonaceous material containing the oligomers are separated from solution and the adsorbed oligomers are desorbed from the carbonaceous material.

Methods for separating carbonaceous material, such as MCM, from solution are well known in the art and include, without limitation, filtering the solution containing the carbonaceous material with a suitable filter or column, centrifuging the solution containing the carbonaceous material, and binding the carbonaceous material to a matrix containing pore sizes and surface areas suitable to bind the carbonaceous material.

Methods of desorbing biomass oligomers from carbonaceous material of the present disclosure, such as MCM, include, without limitation, washing the carbonaceous material containing that oligomers with an ionic liquid, an acid, an alcohol, water, a mixture of an alcohol and water, or a mixture of LiCl and N,N-dimethylacetamide. As disclosed herein, carbonaceous material of the present disclosure, such as MCM, containing adsorbed oligomers are washed under conditions suitable to desorb the oligomers from the carbonaceous material. The ratio of each component in a wash mixture may vary depending on the amount and type of carbonaceous material used, and the amount and type of adsorbed oligomer.

In certain embodiments, the adsorbed oligomers are desorbed by washing the carbonaceous material, such as MCM, with an alcohol, water, or any mixture thereof. Suitable alcohols may include, for example, methanol, ethanol or propanol. A mixture of alcohol and water may also be used for the wash. For example, a mixture of ethanol and water may have a 50:40, 60:40, 70:40, 80:40, or 90:40 volume/volume ratio of ethanol to water. In certain preferred embodiments, the adsorbed oligomers are desorbed by washing the carbonaceous material, such as MCM, with a mixture of ethanol and water having a 60:40 volume/volume ratio of ethanol to water (see, Blum et al. Archives of Biochemistry and Biophysics 1960, 91, 21-26).

It should be understood that desorption of oligomers may further include depolymerizing the adsorbed oligomers into shorter-chain oligomers, such as monomers, and isolating these shorter-chain oligomers. For example, in some embodiments, an oligomer-adsorbed MCM may be treated with an acid-containing solution, which causes the adsorbed oligomer to hydrolyze into a monomer, which subsequently desorbs due to its much weaker adsorption to the MCM relative to longer-chained oligomers. The resulting monomers may be adsorbed on the MCM, solubilized in the acid-containing solution used, or a combination thereof. To isolate some of these monomers, the MCM may be separated from the acid-containing solution by any suitable methods known in the art, such as filtration or centrifugation. The separated MCM may be washed, either with water (e.g., hot water) or acid to further desorb monomers that may remain on the separated MCM. Thus, the desorbed monomers may be found in both the acid-containing solution used, as well as the wash. In certain embodiments, the adsorbed oligomer is a polysaccharide. Examples of suitable polysaccharides include, without limitation, glucan, xylan, and a combination thereof. In other embodiments, the produced monomer is a monosaccharide. Examples of suitable monosaccharides include, without limitation, glucose, xylose, and a combination thereof.

Desorption of oligomers using any of the methods described above may be performed at any suitable temperature. For example, in some embodiments, the temperature is between 10° C. and 200° C., between 10° C. and 80° C., between 100° C. and 150° C., or between 20° C. and 30° C. In other embodiments, the temperature is about 25° C., or about 125° C. The temperature may vary depending on the wash selected. In one embodiment, water may be used for desorbing at elevated temperatures such as between 90° C. and 150° C., or at about 125° C. In another embodiment, acid may be used for desorbing adsorbed oligomers at room temperature (e.g., about 25° C.). In yet another embodiment, acid may be used for desorbing adsorbed oligomers at elevated temperatures, including, for example, between 90° C. and 150° C., or at about 125° C.

### Functionalized Carbonaceous Material

In certain embodiments, carbonaceous material of the present disclosure, such as MCM, contain an acid-functionalized surface, a base-functionalized surface, or an acid/base-functionalized surface that further depolymerizes adsorbed biomass oligomers into shorter chain oligomers.

Methods for functionalizing the surfaces of carbonaceous material, such as MCM, are well known in the art. For example, carbonaceous material may be acid functionalized by oxidation of a carbonaceous material surface using acids or ozone, controlled impregnation of the carbonaceous material with organic monomers that can be subsequently converted to functional monomers or polymers, reaction of carbonaceous material surfaces with diazonium compounds, and reaction of the carbonaceous material surfaces with nitric acid to functionalize the carbonaceous material surface with carboxylic acid (see, Liang et al., Angew. Chem. Int. Ed. 2008, 47, 3696-3717; and Bazula et al., Microporous and Mesoporous Materials, 2008, 108, 266-275). In other examples, carbonaceous material may be base-functionalized by using a Lewis base, including any Lewis base that can become a Bronsted-Lowry base in water. In one example, carbonaceous material may be functionalized with a base such as phenolate at a pH corresponding to the pKa of phenol. Moreover, acid/base-functionalized carbonaceous material may be obtained by further controlled oxidation of base-functionalized carbonaceous material.

Accordingly, any of the carbonaceous material of the present disclosure, such as MCM, may contain an acid-functionalized surface, a base-functionalized surface, or an acid/base-functionalized surface.

In certain embodiments, carbonaceous material, such as MCM, is functionalized with a mineral acid. Suitable mineral acids include, without limitation, sulfonic acids and phosphonic acids. In other embodiments, carbonaceous material, such as MCM, is functionalized with an organic acid. Examples of suitable organic acids include, without limitation, carboxylic acids. In certain preferred embodiments, MCM of the present disclosure contain a phosphonic acid. In particular, MCM containing a phosphonic acid, have a phosphonic acid-functionalized surface.

Acid-functionalized carbonaceous material of the present disclosure, such as MCM, can adsorb lignocellulosic oligomers. Examples of lignocellulosic oligomers adsorbed by acid-functionalized carbonaceous material, such as MCM, include, without limitation, lignin oligomers, hemicellulose oligomers, poly-β-glucan fragments derived from cellulose, oligosaccharides, long chain poly-β-glucans, glucose, cellobiose, cellotriose, cellotetraose, cellopentaose, cellohexaose, xylose, xylans, xyloglucans, mannans, mannose, galactose, rhamnose, arabinose, arabinoxylans, and combinations thereof.

In some embodiments, the adsorption capacity of the acid-functionalized carbonaceous material, such as MCM, for oligomers is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more of oligomer mass uptake relative to the mass of the acid-functionalized carbonaceous material. In other embodiments, the adsorption capacity of acid-functionalized carbonaceous material, such as MCM, for oligomers ranges from at least 40% to at least 95% of oligomer mass uptake relative to the mass of the acid-functionalized carbonaceous material.

In other embodiments, the acid-functionalized carbonaceous material, such as MCM, adsorbs oligomers at a pH of about 7.0, about 6.5, about 6.0, about 5.5, about 5.0, about 4.5, about 4.0, about 3.5, about 3.0, about 2.5, about 2.0, about 1.5, about 1.0, about 0.5, or about 0. In still other embodiments, the acid-functionalized carbonaceous material, such as MCM, adsorbs oligomers at a temperature of at least 15° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., at least 100° C., or higher. In certain preferred embodiments, the acid-functionalized carbonaceous material, such as MCM, adsorbs oligomers at a temperature that ranges from about 20° C. to about 30° C.

In some embodiments, carbonaceous material, such as MCM, is functionalized with hydroxyl-containing groups. In other embodiments, carbonaceous material, such as MCM, is functionalized with a Lewis base. In one embodiment, carbonaceous material, such as MCM, may be functionalized with phenolate or carboxylate.

In yet other embodiments, carbonaceous material, such as MCM, is dual functionalized. It should be understood that “base” may include a conjugate base relative to acid functionality. For example, in one embodiment, carbonaceous material, such as MCM, may be functionalized with carboxylic acid and carboxylate. In another example, carbonaceous material, such as MCM, may be functionalized with phenolic acid and phenolate. In certain embodiments, the acid and base may be present in equimolar amounts in the carbonaceous material, such as MCM. In other embodiments, the acid may be present in excess of the base, or the base may be present in excess of the acid.

In certain embodiments, the shorter chain oligomers produced by the further depolymerization of adsorbed biomass oligomers are monosaccharides. In certain preferred embodiments, the produced monosaccharide is glucose, xylose, or a combination thereof.

In other embodiments, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more of the adsorbed oligomers are depolymerized to glucose by acid-functionalized carbonaceous material of the present disclosure, such as MCM.

In further embodiments, once the acid-functionalized carbonaceous material, such as MCM, has adsorbed the oligomers from solution, the acid-functionalized carbonaceous material containing the oligomers is separated from solution and the adsorbed oligomers are desorbed from the acid-functionalized carbonaceous material.

In some embodiments, the acid-functionalized carbonaceous material, such as MCM, is separated from solution by filtering the solution containing the acid-functionalized carbonaceous material with a suitable filter or column, centrifuging the solution containing the acid-functionalized carbonaceous material, or binding the acid-functionalized carbonaceous material to a matrix containing pore sizes and surface areas suitable to bind the acid-functionalized carbonaceous material.

In other embodiments, the oligomers adsorbed on the acid-functionalized carbonaceous material, such as MCM, are desorbed by washing the acid-functionalized carbonaceous material containing that oligomers with an ionic liquid, an acid, an alcohol, water, a mixture of an alcohol and water, or a mixture of LiCl and N,N-dimethylacetamide. For example, the alcohol may be ethanol. In certain embodiments, the adsorbed oligomers are desorbed by washing the acid-functionalized carbonaceous material, such as MCM, with a mixture of ethanol and water having a 60:40 volume/volume ratio of ethanol to water.

In some embodiments, the shorter chain oligomers produced by the further depolymerization of adsorbed biomass oligomers are desorbed from the carbonaceous material, such as MCM, with, for example, an ionic liquid, an acid, an alcohol (e.g., ethanol), water, a mixture of an alcohol and water, or a mixture of LiCl and N,N-dimethylacetamide. In other embodiments, the shorter chain oligomers produced by the further depolymerization of adsorbed biomass oligomers are monosaccharides, including without limitation, glucose, xylose, and a combination thereof. In certain embodiments, the monosaccharides are desorbed from the carbonaceous material, such as MCM, with, for example, an ionic liquid, an acid, an alcohol (e.g., ethanol), water, a mixture of an alcohol and water, or a mixture of LiCl and N,N-dimethylacetamide.

### Applications

The carbonaceous materials, including the MCM and functionalized MCM (e.g., acid-functionalized, base-functionalized, acid/base-functionalized MCM), may have various industrial applications. One such application is the separation of five-carbon and six-carbon sugars that may be produced from hydrolysis of biomass containing a mixture of hemicellulose and cellulose.

For example, hydrolysis of cellulosic biomass using a mineral acid typically results in a mixture of pentose and hexose; however, pentose and hexose monomers can be difficult to separate downstream. The carbonaceous materials described herein, and the methods employing such carbonaceous materials, provide a method for separating five-carbon and six-carbon sugars, such as pentose and hexose respectively, upstream. Such method takes advantage of (1) the higher kinetic rate of hydrolysis of hemicellulose (C5-polymer) compared to cellulose (C6-polymer); and (2) the ability of MCM to selectively adsorb carbohydrate polymer mixtures with high affinity versus monomers.

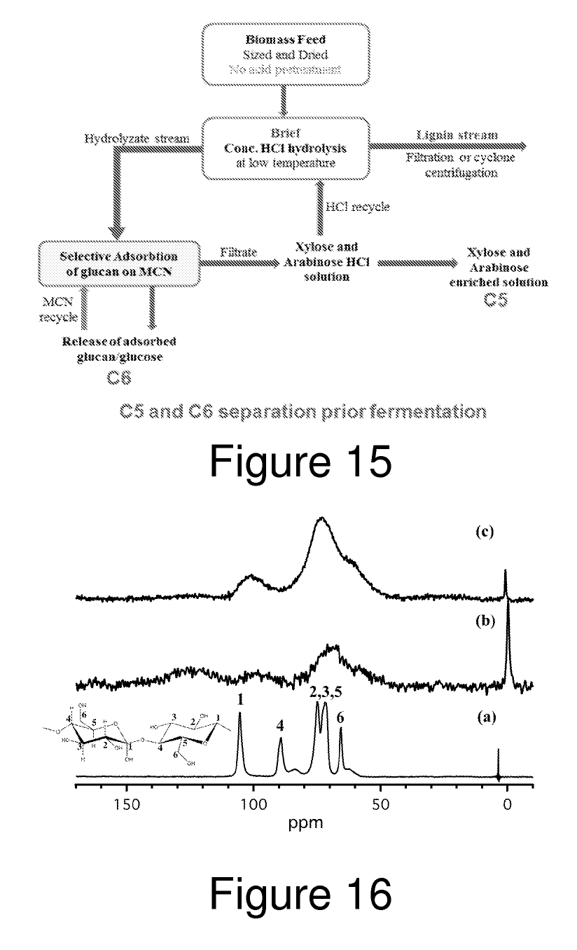

With reference to FIG. 15, an exemplary process for separating C6 oligomers from C5 oligomers by selectively adsorbing glucan on the MCN. It should be understood that “C6 oligomers” and “C5 oligomers” refers to monomers with six or five carbon atoms, respectively, or polymers where their monomeric units have six or five carbon atoms, respectively. Examples of C6 oligomers include glucan and glucose. Examples of C5 oligomers include xylose and arabinose.

### Biofuel Production

Certain aspects of the present disclosure relate to the use of purified biomass oligomers (e.g., lignocellulosic and/or cellulosic oligomers) produced by any of the methods of the present disclosure, in the production of biofuels, such as ethanol or butanol.

Ethanol can be produced by enzymatic conversion (e.g., fermentation) of the produced lignocellulosic and/or cellulosic oligomers. This kind of ethanol is often referred to as bioethanol or biofuel. It can be used as a fuel additive or extender in blends of from less than 1% and up to 100% (a fuel substitute). Biofuels may be produced by converting the lignocellulosic and/or cellulosic oligomers produced by the methods of the present disclosure to biofuel (e.g., ethanol) by any technique known in the art including, without limitation, microbial or chemical fermentation, and biological lipid synthesis.

Fermentative microorganisms may be any microorganism suitable for use in a desired fermentation product synthesis process. Suitable microorganisms are able to convert lignocellulosic and/or cellulosic oligomers, such as oligosaccharides, glucose, xylose, arabinose, mannose, or galactose directly or indirectly into the desired biofuel. Suitable fermentative microorganisms include, without limitation, yeast, fungi, algae, bacteria, and combinations thereof. Non-limiting examples include Saccharomyces spp., Corynebacterium spp., Brevibacterium spp., Rhodococcus spp., Azotobacter spp., Citrobacter spp., Enterobacter spp., Clostridium spp., Klebsiella spp., Salmonella spp., Lactobacillus spp., Aspergillus spp., Zygosaccharomyces spp., Pichia spp., Kluyveromyces spp., Candida spp., Hansenula spp., Dunaliella spp., Debaryomyces spp., Mucor spp., Torulopsis spp., Methylobacteria spp., Bacillus spp., Escherichia spp., Pseudomonas spp., Serratia spp., Rhizobium spp., and Streptomyces spp., Zymomonas mobilis, acetic acid bacteria (family Acetobacteraceae), methylotrophic bacteria, Propionibacterium, Acetobacter, Arthrobacter, Ralstonia, Gluconobacter, Propionibacterium, and Rhodococcus.

It is to be understood that while the present disclosure has been described in conjunction with the preferred specific embodiments thereof, the foregoing description is intended to illustrate and not limit the scope of the present disclosure. Other aspects, advantages, and modifications within the scope of the present disclosure will be apparent to those skilled in the art to which the present disclosure pertains.

The following examples are offered to illustrate provided embodiments and are not intended to limit the scope of the present disclosure.

### EXAMPLES

### Example 1

### Synthesis of Mesoporous Carbon Nanoparticles

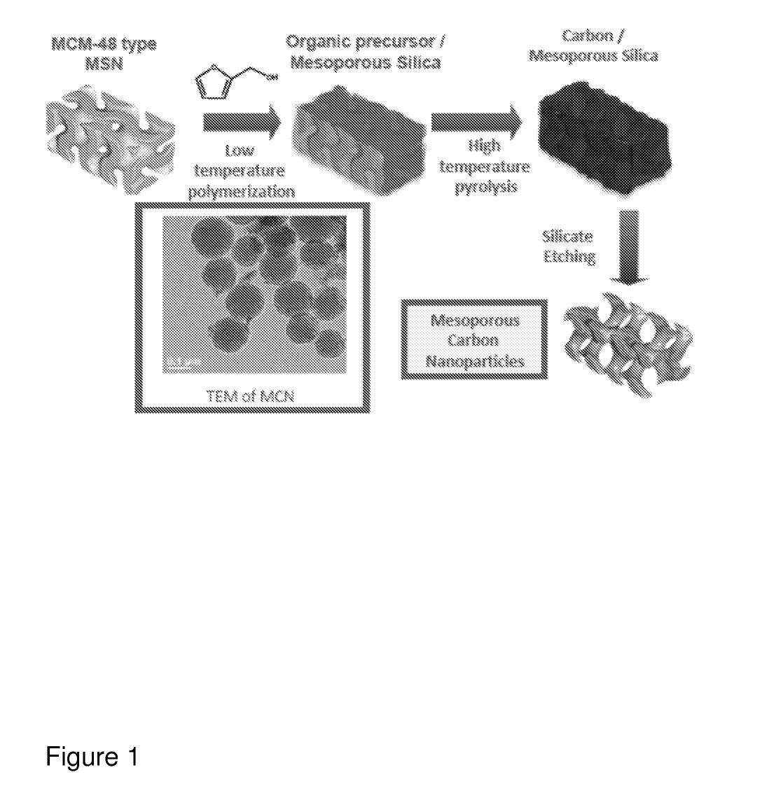

A schematic of the synthesis of 100 nm sized mesoporous carbon nanoparticles (MCN) is shown in FIG. 1.

To prepare the MCN material, MCM-48 type mesoporous silica nanoparticles (MSN) were first synthesized as the structure-directing template via a modified Stöber method. Cetyltrimethylammonium bromide (CTAB; 1.0 g) and a triblock copolymer (Pluronic F 127, EO₁₀₆PO₇₀EO₁₀₆; 4.0 g) were mixed in 298 mL of H₂O/NH₃/EtOH solution (NH₄OH)_{aq} (2.8 wt %)/EtOH=2.5/1 (v/v)). Tetraethyl orthosilicate (TEOS; 3.6 g) was added into the solution at room temperature. After vigorous stirring for 1 min, the reaction mixture was kept under static condition for 1 day at room temperature for the complete condensation of silica. The resulting solid MSN product was isolated by centrifuge, washed with copious water, and dried at 70° C. in air.

To synthesize mesoporous carbon nanoparticles, the surface of the MSN were first converted to an aluminosilicate form. As-synthesized MCM-48 material was calcined at 550° C. to remove the surfactant. The calcined sample was then mixed with distilled water to make surface silanol groups, and then completely dried at 150° C. The dried sample was then slurried in an ethanol solution of anhydrous AlCl₃ (Si/Al=20). The ethanol solvent was completely evaporated by rotary evaporator. The dried sample was calcined again at 550° C.

Mesoporous carbon nanoparticles were prepared using furfuryl alcohol (Aldrich) as a carbon source. 1 g of aluminated MCM-48 nanoparticles were infiltrated with 0.91 mL of furfuryl alcohol by impregnation method. The mixture was moved into Schlenk reactor, and subjected to freeze-vacuum-thaw three times using liquid N₂. The mixture was kept under vacuum at 35° C. for 1 hr. After opening the Schlenk reactor, the mixture was heated for 6 hr at 100° C. for polymerization of furfuryl alcohol, and then partially carbonized at 350° C. for 3 hr under vacuum. After cooled to room temperature, the sample was added to 0.58 mL of furfuryl alcohol, and the freeze-vacuum-thaw and polymerization was repeated. Further carbonization was accomplished by heating to 900° C. under vacuum conditions. The carbon product was then collected by HF washing. (10 wt % HF in EtOH/H₂O solution). The aluminated MSN was then infiltrated by furfuryl alcohol at room temperature, followed by polymerization and carbonization at elevated temperatures under vacuum. The silica template was then removed by washing the composite with HF(aq) to yield the desired mesoporous carbon nanoparticles.

### Example 2

### Adsorption of Glucose on MCN

Aqueous solutions of glucose at both pH 7 and pH 0 were prepared utilizing various glucose concentrations (250 g/L, 200 g/L, 125 g/L, 100 g/L, 50 g/L). 0.3 mL of each glucose solution was then treated with 20 mg of MCN and the mixture was vortexed for 24 hr at room temperature to reach equilibrium. After the 24 hr equilibration period, the solution was filtered with a 200 nm PTFE membrane syringe filter. The concentration of the filtrate was then measured by HPLC.

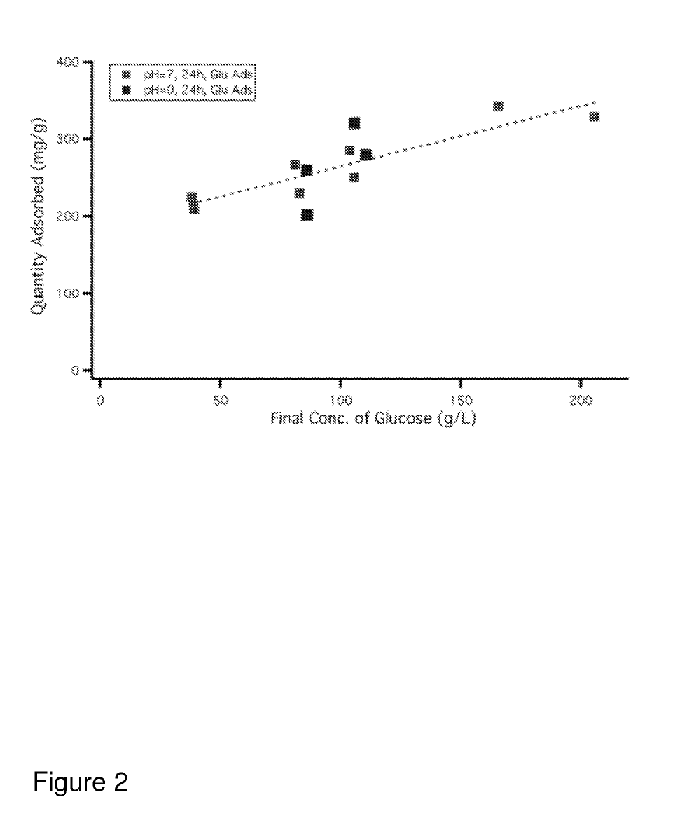

The adsorption capacity of the MCN for glucose was determined to be 300 mg per gram of MCN at a final glucose concentration of 200 g/L (FIG. 2). This adsorption capacity is over 1.5-fold higher than that of conventional activated carbon. Moreover, as shown in FIG. 2, the adsorption capacity of MCN for glucose was similar at both low pH (pH 0) and neutral pH (pH 7) (FIG. 2).

### Example 3

### Adsorption of Cellobiose on MCN

Aqueous solutions of cellobiose at both pH 7 and pH 0 were prepared utilizing various cellobiose concentrations (120 g/L, 100 g/L, 60 g/L, 25 g/L, 10 g/L). 0.3 mL of each solution was then treated with 10 mg of MCN and the mixture was vortexed for 24 hr to reach equilibrium. After the 24 hr equilibration period, the solution was filtered with a 200 nm PTFE membrane syringe filter. The concentration of the filtrate was then measured by HPLC.

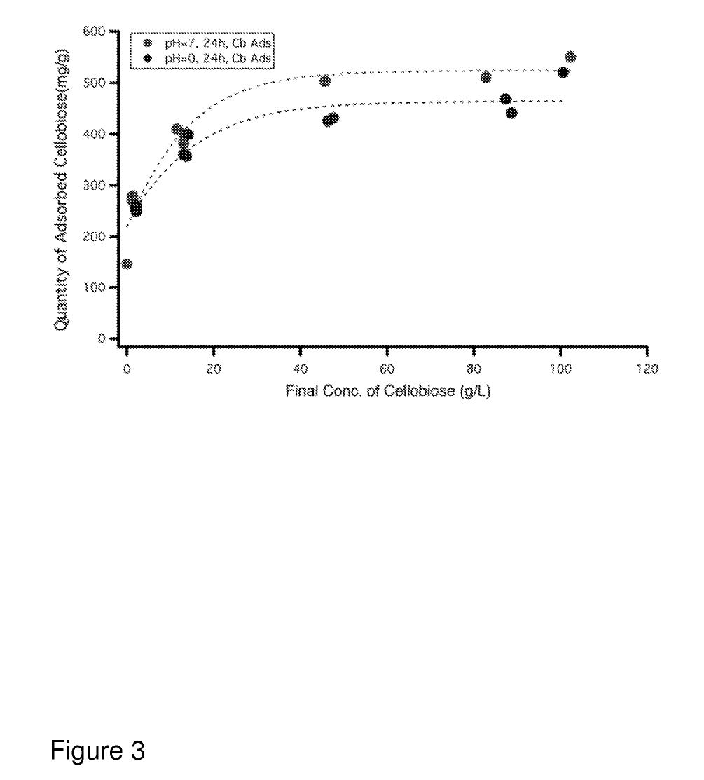

The adsorption capacity of the MCN for cellobiose was determined to be 500 mg per gram of MCN at a final cellobiose concentration of 60 g/L or more at pH 7 (FIG. 3). This adsorption capacity is approximately 50% of mass uptake relative to the mass of MCN. The adsorption capacity of MCN for cellobiose at pH 0 was 440 mg per gram of MCN at a final cellobiose concentration of 60 g/L or more (FIG. 3). The adsorption capacity at pH 0 represents a decrease of less than 12% compared to the adsorption capacity at pH 7. These results demonstrate that MCN has a similar coefficient of binding at both low pH (pH 0) and neutral pH (pH 7).

### Example 4

### Adsorption of Oligosaccharides on MCN

An equimass aqueous solution of oligosaccharides was prepared by combining 40 mg/L of each of glucose (Glc), cellobiose (Cb), cellotriose (G3), cellotetraose (G4), cellopentaose (G5), and cellohexaose (G6). 1 mL of the oligosaccharide solution was then treated with 5 mg of MCN at room temperature for 10 min. The treated solution was then filtered with a 1 mL speedisc column (JT Baker JTB #8163-01) and a vacuum chamber assembly. The filtrate containing the MCN was then washed with 15 mL of warm water followed by 15 mL of a warm ethanol/water mixture (60/40, v/v). The MCN was then dried, rehydrated for dilution, and analyzed by Dionex HPLC.

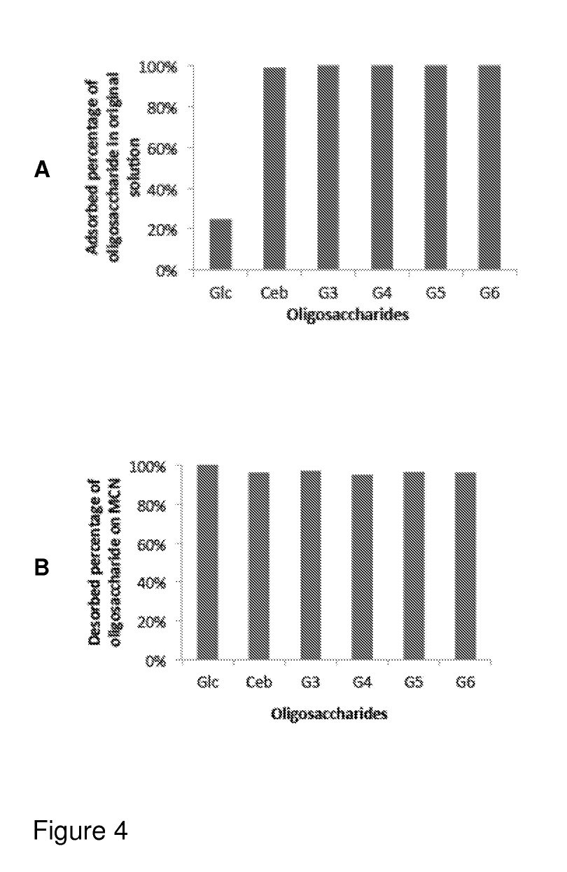

As shown in FIG. 4, the MCN had a nearly quantitative efficiency for oligosaccharide (Glc, Cb, G3, G4, G5, and G6) adsorption. Moreover, washing the adsorbed oligosaccharides with 15 mL of water, followed by 15 mL of the ethanol/water mixture desorbed almost 100% of the oligosaccharides from the MCN (FIG. 4).

### Example 5

### Synthesis of an Acid-Treated Cellulose Solution

An acid-treated cellulose solution was synthesized by: (i) treating 10 mg of Avicel with 5 mL of concentrated HCl (37% aqueous HCl) at room temperature; (ii) vortex mixing the solution for 30 sec; (iii) adding 15 mL of concentrated HCl (37% aqueous HCl) at −20° C. until the mixture rapidly dissolves; (iv) vortex mixing the resulting solution for 30 sec; and (v) incubating the solution for 10 min at −20° C. The cellulose solution was then hydrolyzed by incubating the solution with concentrated HCl (37%) at room temperature for 2-96 hr. The resulting hydrolyzed solution was then diluted and the concentrations of the glucose (Glc), cellobiose (Cb), cellotriose (G3), cellotetraose (G4), cellopentaose (G5), and cellohexaose (G6) produced by the hydrolysis was analyzed by Dionex HPLC.

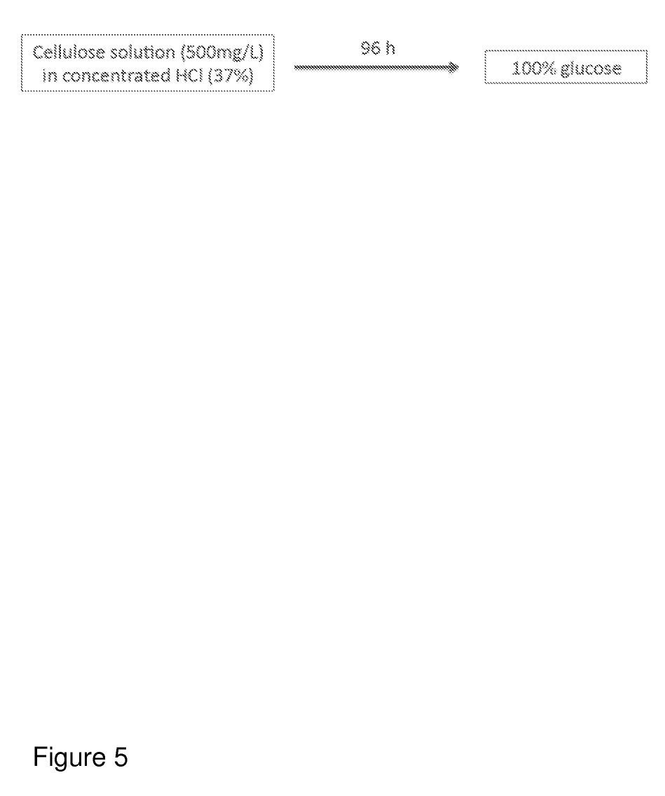

As shown in FIG. 5 and Table 2, 500 mg/mL of the cellulose solution treated with concentrated HCl (37%) was fully hydrolyzed into glucose (100%) after 96 hr at room temperature. These results are similar to those reported in U.S. Pat. No. 5,972,118.

TABLE 1
Cellulose hydrolysis after 96 h at room temperature

| | Products | | |
|---|---|---|---|
| | Glucose | G2-G6 | G7 |
| Product yields (%) | 100 | 0 | 0 |

### Example 6

### MCN Adsorption of Poly-(3-Glucans Derived from Cellulose

An acid-treated cellulose solution was synthesized as described in Example 5 above. The cellulose solution was then hydrolyzed by incubating the solution with concentrated HCl (37%) at room temperature for 2 hr. 1 mL of the hydrolyzed cellulose solution was then treated with 5 mg of MCN for 10 min at 4° C. The mixture was then filtered with a 1 mL speedisc column (JT Baker JTB #8163-01) and a vacuum chamber assembly, and the resulting filtrate was washed with 2 mL of water. The filtrate was then diluted and the concentrations of the glucose (Glc), cellobiose (Cb), cellotriose (G3), cellotetraose (G4), cellopentaose (G5), and cellohexaose (G6) produced by the hydrolysis was analyzed by Dionex HPLC.

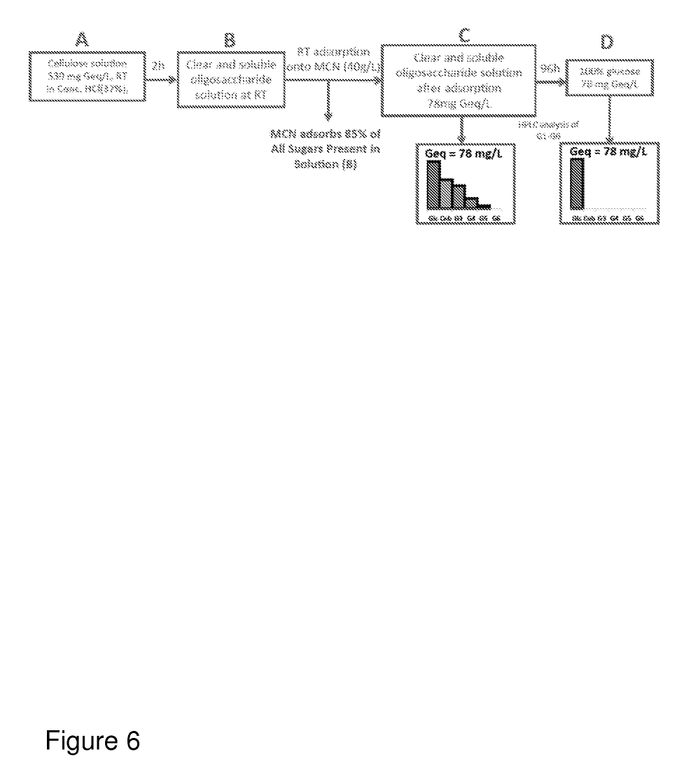

As shown in FIG. 6, the MCN adsorbed 85% of all the sugars produced by the hydrolysis of the cellulose solution. Moreover, 78% of the oligosaccharides adsorbed on the MCN were longer than G6 and therefore could not be quantified by Dionex HPLC in aqueous solution (FIG. 6). These results demonstrate that MCN can be used to remove a large fraction of sugars present in solution and that most of the sugars can be present in the form of long-chain oligoglucans that are insoluble in water.

### Example 7

### HPLC Analysis of Cellulose Solution after Hydrolysis

An acid-treated cellulose solution was synthesized as described in Example 5 above. The cellulose solution was then hydrolyzed by incubating the solution with concentrated HCl (37%) at room temperature for 2 hr. The cellulose solution was then hydrolyzed by incubating the solution with concentrated HCl (37%) at room temperature for 2 hr. The resulting hydrolyzed solution was then diluted and the concentrations of the glucose (Glc), cellobiose (Cb), cellotriose (G3), cellotetraose (G4), cellopentaose (G5), and cellohexaose (G6) produced by the hydrolysis was analyzed by Dionex HPLC.

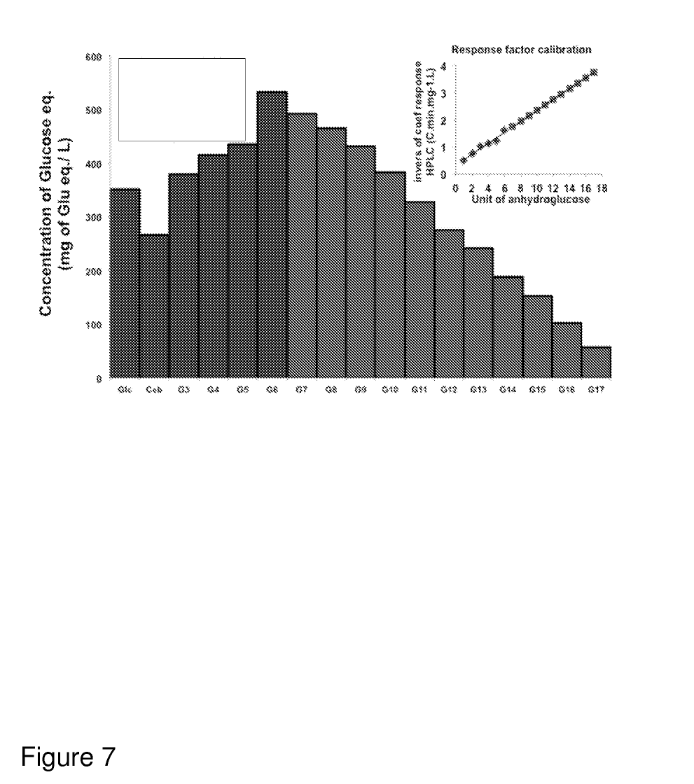

HPLC analysis showed that the cellulose solution resulting from the hydrolysis contained higher glucose oligomers, many of which (those above G6) were insoluble in aqueous solution (FIG. 7).

### Example 8

### MALDI-TOF-MS of Adsorbed Higher Oligosaccharides

An acid-treated cellulose solution was synthesized as described in Example 5 above. The cellulose solution was then hydrolyzed by incubating the solution with concentrated HCl (37%) at room temperature for 2 hr. 1 mL of the hydrolyzed cellulose solution was then treated with 5 mg of MCN for 10 min at 4° C. The mixture was then filtered and the filtrate was then washed with 2 mL of water. The filtrate was then mixed with 1 μL of 65 mM 2,5-dihydroxybenzoic acid (DHB) in a 0.65:0.35 v:v acetonitrile:water solution. The mixture was then dried on a sample plate. MALDI-TOF-MS was then performed using a Shimadzu Axima Performance instrument.

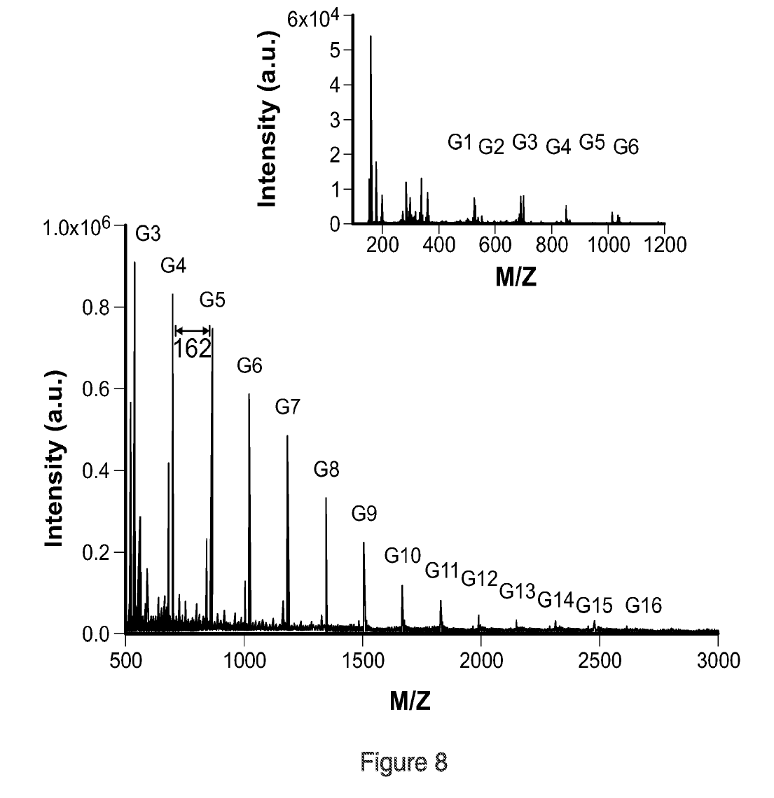

As shown in FIG. 8, the MALDI-TOF-MS showed that higher oligosaccharides (i.e., greater than G6) produced from the hydrolysis of the cellulose solution were adsorbed on.

### Example 9

### Synthesis of Sulfonic Acid-Functionalized Mesoporous Carbon Nanoparticles

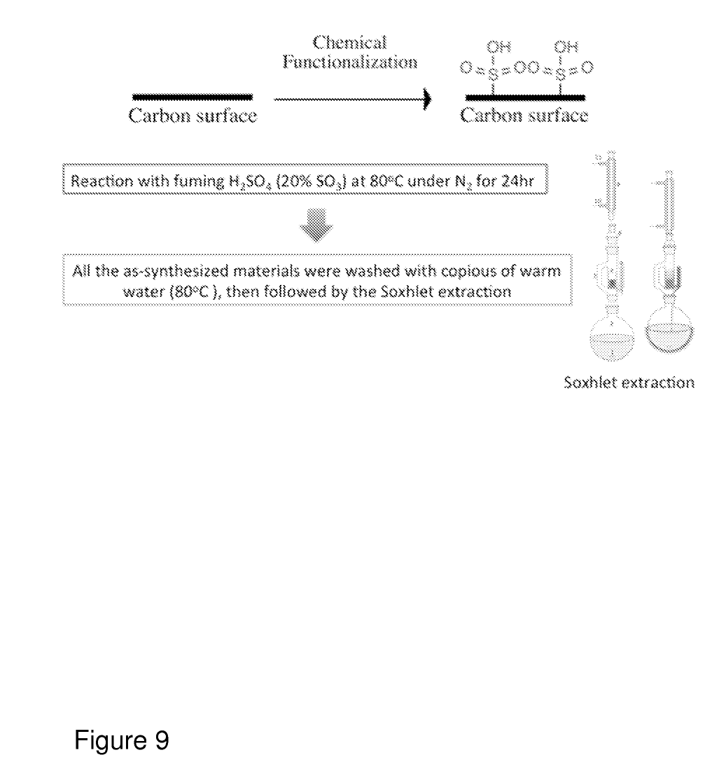

One gram of MCN was reacted with 21.4 mL of fuming H₂SO₄ (20% SO₃) at 80° C. under N₂ for 24 hr. After 24 hr, the reaction was quenched by pouring the solution into one liter of 4° C. cold water. The solid carbon material was then collected via filtration, followed by washing with 2 L of 80° C. warm water. The as-synthesized material was washed four times via Soxhlet extraction using 250 mL of water, for a period of 12 hr for each wash, until there was no trace of the sulfate anion, as measured by the BaCl₂ method. This method relies on preparing a 1M batch of BaCl₂ solution for the detection of sulfate anion. After Soxhlet extraction, 1 mL of the extracted solution was mixed with 1 mL of the 1M BaCl₂ solution. After mixing, a white precipitate forms instantaneously if there is a trace of sulfate anion. A schematic of the procedure is shown in FIG. 9.

### Example 10

### pH and Acid Leaching Analysis of Sulfonated MCN

The sulfonated MCN(SO₃-MCN) was compared to Amberlyst 15 and Nafion NR50. 25 mg of SO₃-MCN, Amberlyst 15, and Nafion NR50 were each mixed with 1 mL of water and stirred for 30 min. After 30 min of equilibration time, each of the solutions was filtered and 1 mL of the filtrate was mixed with 1 mL of 1M BaCl₂ solution. After mixing, a white precipitate will form instantaneously if there is a trace of sulfate anion.

As shown in Table 2, SO₃-MCN and Nafion NR50 did not leach acid, even when treated under extended boiling water conditions of Soxhlet extraction.

TABLE 2
pH Measurement

| | Acid catalyst | | |
|---|---|---|---|
| | SO₃-MCN | Amberlys 15 | Nafion NR50 |
| pH with particles | 2.9 | 3.3 | 3.7 |
| pH of filtrate | 5.2 | 3.7 | 5.7 |
| BaCl₂ treatment | clear | white precipitate | clear |

### Example 11

### Conversion of Cellobiose by Sulfonated MCN

An aqueous cellobiose solution (1 mL of 10 g/L Cb) was prepared. The cellobiose solution was then treated with 25 mg of SO₃-MCN. The reaction was conducted at 125° C. using different reaction times. After the reaction, the solution was filtered with a 1 mL speedisc column (JT Baker JTB #8163-01) and a vacuum chamber assembly, and the cellobiose-adsorbed filtrate was washed with warm water and a warm ethanol/water solution (60:40 v:v), in an alternating fashion. The solvent was then completely removed by rotary evaporation. The resulting product was redissolved in 10 mL of water and analyzed via HPLC. The concentration of both cellobiose and glucose was analyzed by a Shimadzu HPLC system.

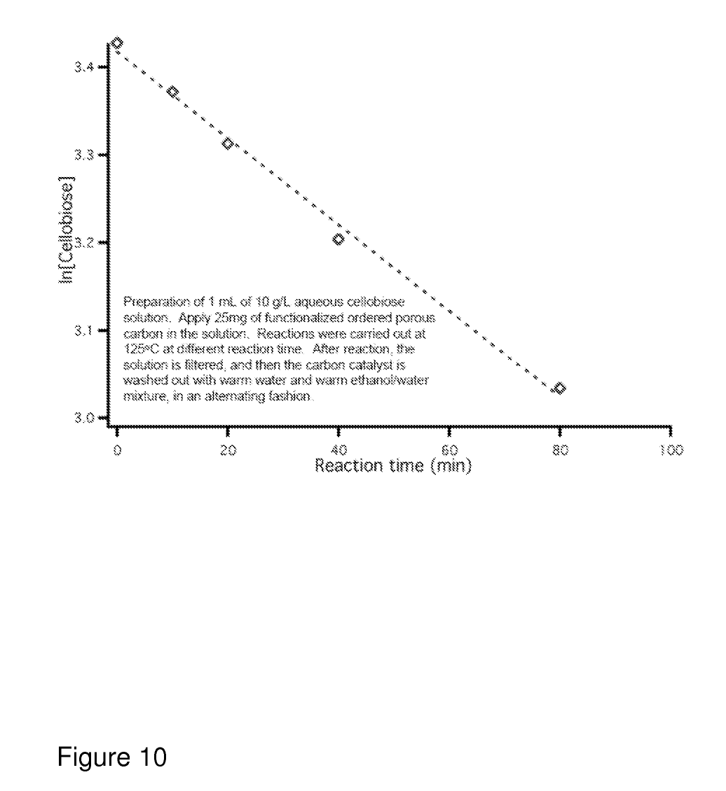

A plot of the log of cellobiose concentration as a function of reaction time shows that the SO₃-MCN catalyzed the hydrolysis of cellobiose as a first-order reaction (FIG. 10).

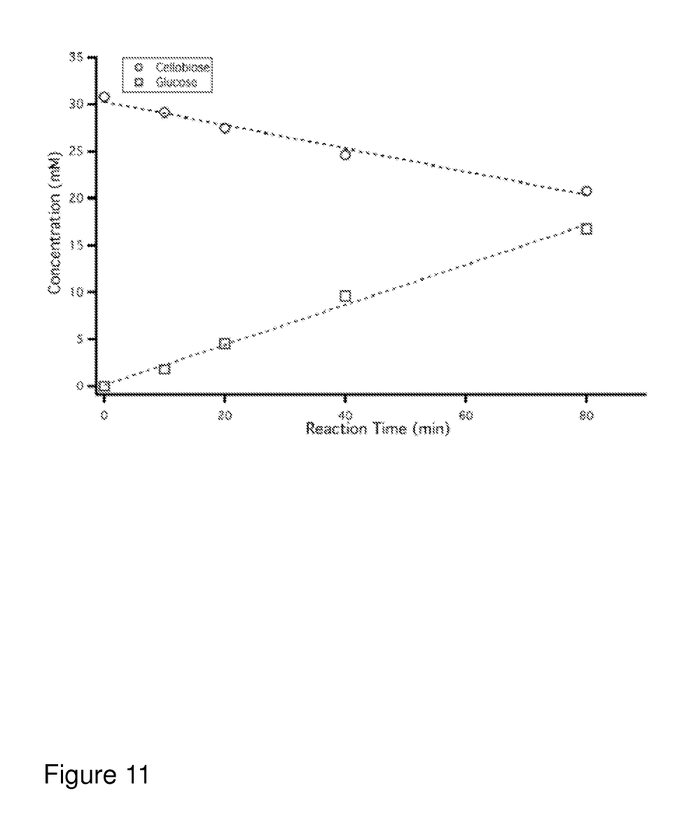

Moreover, the selectivity of cellobiose hydrolysis to glucose catalyzed by SO₃-MCN reached 87% (FIG. 11).

### Example 12

### Hydrolysis of Cellulose Catalyzed by SO₃-MCN

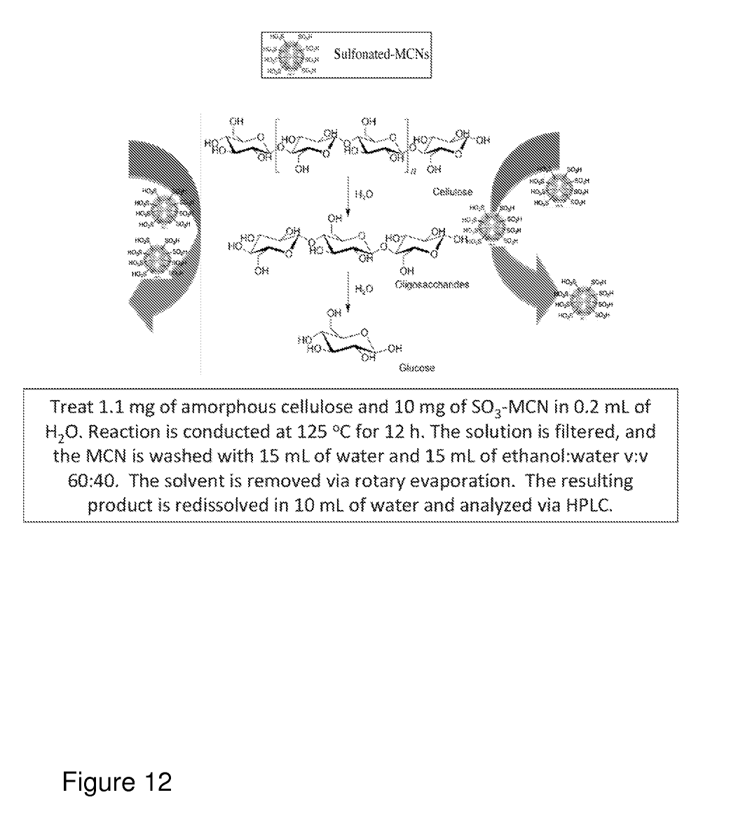

1.1 mg of amorphous cellulose was mixed with 10 mg of SO₃-MCN in 0.2 mL of water at 125° C. for 12 hr. After the 12 hr incubation, some cellulose still remained unreacted. The mixture was then filtered with a 1 mL speedisc column (JT Baker JTB #8163-01) and a vacuum chamber assembly, and the SO₃-MCN was washed with 15 mL of water and 15 mL of an ethanol:water v:v 60:40 solution. The solvent was then removed by rotary evaporation. The resulting product was redissolved in 10 mL of water and analyzed by HPLC. A schematic of the procedure is shown in FIG. 12.

The results of the HPLC analysis showed that the glucose yield was 2.7%, the oligosaccharide (G2-G6) yield was 19%, and the high oligosaccharide (>G7⁺) yield was greater than 0 (Table 3). These results demonstrate that SO₃-MCN is an active catalyst for depolymerizing cellulose in water.

TABLE 3
HPLC analysis

| | Product(s) | | |
|---|---|---|---|
| | Glucose | G2-G6 | G7 |
| Product yield (%) | 2.7 | 19 | >0 |

### Example 13

### Synthesis of Phosphonic-Acid Functionalized Mesoporous Carbon Material

SBA-15 was synthesized according to Stucky method (see, Zhao et al., J. Am. Chem. Soc., 1998, 120, 6024), except that the resulting material was calcined at 800° C. in order to simultaneously remove P123 surfactant and dehydroxylate the surface. The pores of the SBA-15 template were completely or partially filled by adding a 60 wt % aqueous solution of furfural to a given amount of SBA-15. This was performed by adding 0.96 mL of 60 wt % aqueous furfural solution to 1.5 g of SBA-15 template and homogenizing according to the procedure reported by Antonietti et al. (see, Antonietti et al. J. Mater. Chem., 2007, 17, 3412). The resulting furfural/SBA-15 wet powder was placed inside a 25 mL capacity Teflon-lined stainless steel autoclave, which was heated in an oven at 180° C. for 24 hr. The resulting products were filtered, washed with water and methanol, and dried in a vacuum oven at 60° C. for 4 hr. The silica was then removed by etching from the composite material, using 15 mL of a 4 M aqueous solution of ammonium hydrogen difluoride (NH₄HF₂), to yield mesoporous carbon replicas.

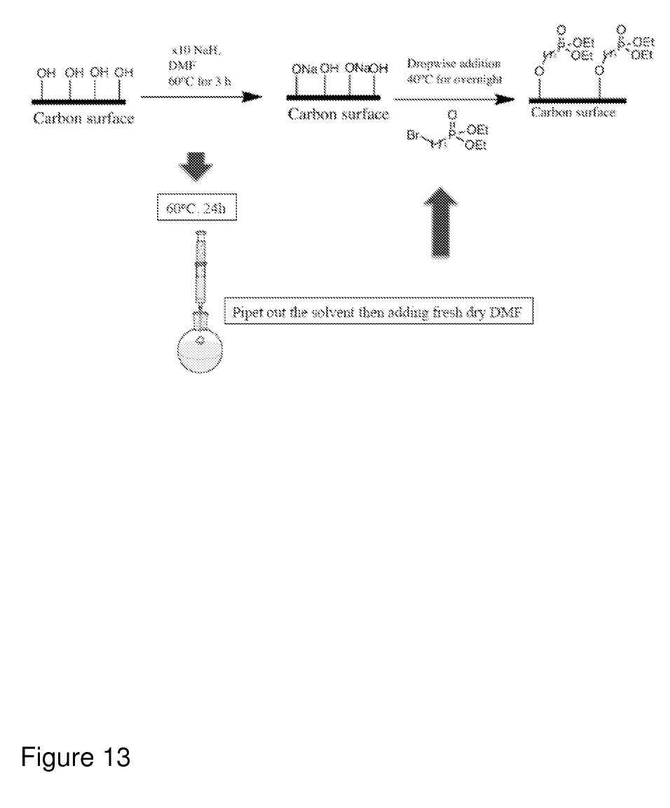

To phosphonate the mesoporous carbon material, 500 mg of the mesoporous carbon material was treated with 800 mg of NaH in 5 mL of DMF. The solution was maintained at 60° C. for 3 hr. 1 mL of diethyl(3-bromopropyl)phosphonate was then added and the solution was treated at 40° C. 12 hr. The resulting phosphonate-functionalized mesoporous carbon material was collected by filtration. A schematic of this procedure is shown in FIG. 13.

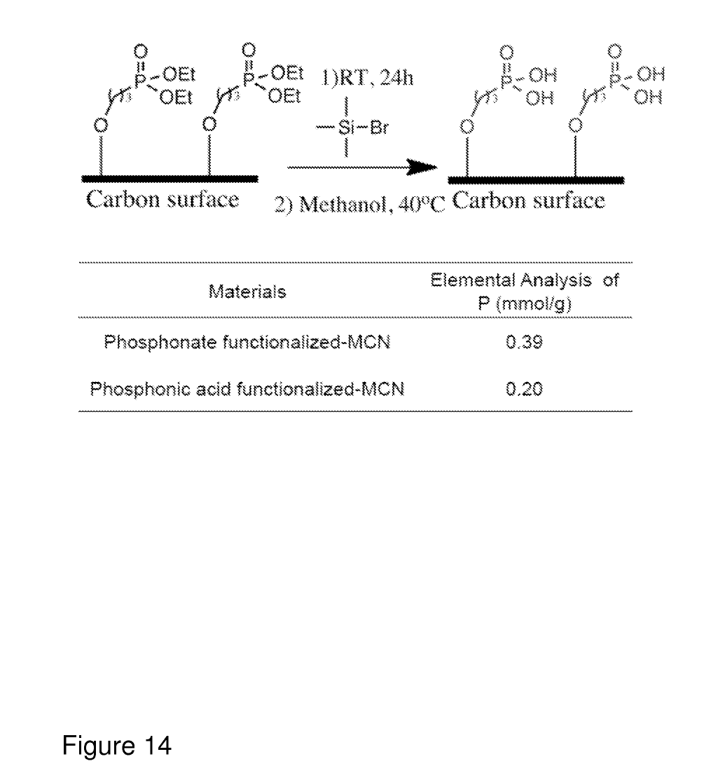

The phosphonate-functionalized mesoporous carbon material was then treated with 2 mL of trimethylbromosilane, and the solution was incubated with stirring at room temperature for 24 hr. 5 mL of methanol were then added, and the resulting solution was incubated at 40° C. for 4 hr. After filtration and collection of the phosphonic-acid functionalized mesoporous carbon material, it was washed with 10 mL of methanol, followed by 10 mL of hexane, and dried under vacuum for 8 hr to remove solvent. This procedure and the coverage of phosphonic acid functionality, as measured by elemental analysis, are shown in FIG. 14.

### Example 14

### Effect of Chain Length and Mesoporosity for Glucan Adsorption on Mesoporous Carbon Nanoparticles

This example demonstrates the effect of glucan chain length on adsorption energetics. Further, the results of this example help with understanding the adsorption of long-chain glucans onto mesoporous carbon nanoparticles (MCN) from a concentrated acid solution, and the effect of mesoporosity on this process. Additionally, adsorption of long-chain glucans in this example is characterized using multiple experimental techniques including HPLC, GPC, MALDI-TOF-MS and solid-state NMR spectroscopy.

Materials and Methods

Synthesis of Mesoporous Carbon Nanoparticles (MCN).

MCN were synthesized using a MCM-48-type mesoporous silica nanoparticle (MSN) material as the structure-directing template. The synthesis was accomplished by mixing cetyltrimethylammonium bromide (CTAB; 1.0 g) and a triblock copolymer (Pluronic F 127, EO₁₀₆PO₇₀EO₁₀₆; 4.0 g) in 298 mL of H₂O/NH₃/EtOH solution NH₄OH(aq) (2.8 wt % NH₄OH in water)/EtOH=2.5/1 (v/v)). Tetraethyl orthosilicate (TEOS; 3.6 g) was added to the solution at room temperature. After vigorous stirring for 1 min, the reaction mixture was kept under a static condition for 1 day at room temperature in order to facilitate silica condensation. The resulting solid MSN product was isolated by centrifugation and washed with copious amounts of water, followed by drying at 70° C. in air. To synthesize the MCN material, the surface of MSN was first converted to an aluminosilicate form. This was performed by first calcining the as-synthesized dry MSN product at 550° C. for a period of 2 h at atmosphere in order to remove the surfactant. The calcined sample was mixed with distilled water to synthesize surface silanol groups, and then it was completely dried at 150° C. in air (atmospheric pressure). The dried sample was slurried in an ethanol solution of anhydrous AlCl₃ (Si/Al=20) for a period of 1 h at room temperature. The ethanol solvent was then completely evaporated via rotary evaporation. The dried sample was calcined again at 550° C. for a period of 2 h in air at atmospheric pressure. Mesoporous carbon nanoparticles were synthesized by using furfuryl alcohol (Aldrich) as the carbon source. 1 g of aluminosilicate MCM-48 nanoparticles were impregnated with 0.91 mL of furfuryl alcohol. The resulting impregnated material was placed into a Schlenk reactor, and was subjected to three freeze-vacuum-thaw degas cycles using liquid N₂. After three freeze-vacuum-thaw cycles, the mixture was kept under vacuum (under 1 mbar) at 35° C. for 1 h to homogeneously distribute the furfuryl alcohol into the pores. After opening the Schlenk reactor, the reactor was maintained at a temperature of 100° C. for 6 h in air (at atmospheric pressure), during which time polymerization of furfuryl alcohol occurred. Then the composite material was transferred to a suitable quartz boat, and subsequently maintained at 350° C. for 3 h under vacuum (1 mbar) in order for partial carbonization to occur. Afterwards, the resulting composite material was impregnated with an additional 0.58 mL of furfuryl alcohol and transferred to the Schlenk reactor. The same aforementioned three freeze-vacuum-thaw degas cycles and polymerization procedure were repeated again. The composite material was again transferred to a quartz boat, and further carbonization was accomplished by heating the reactor to 900° C. for 2 h under vacuum (1 mbar). The carbon product was collected, after dissolving the silica template of the composite material following the last carbonization. This dissolution was accomplished using HF at room temperature for a period of 1 h (10 wt % HF in EtOH/H₂O solution HF (48%)/EtOH/H₂O=20 mL/40 mL/40 mL), followed by washing with copious of water and ethanol.

Adsorption of Glucose and Cellobiose on MCN.

Standard glucose solutions were prepared in pH=7 aqueous solution at varying concentrations (250 g L⁻¹, 200 g L⁻¹, 125 g L⁻¹, 100 g L⁻¹, 50 g L⁻¹). Standard cellobiose solutions were prepared at both pH=7 and pH=0 aqueous solution, also at varying concentrations (120 g L⁻¹, 100 g L⁻¹, 60 g L⁻¹, 25 g L⁻¹, 10 g L⁻¹). The adsorption isotherms were measured after equilibration using a static method. Mesoporous carbon nanoparticles (MCN) (1 g) were Soxhlet extracted with 250 mL water for a period of 3 h, and this extraction procedure was repeated four times. Preweighed amounts of MCN were placed in 1.5 mL Eppendorf tubes with 0.3 mL of sugar solution (20 mg of MCN was used for glucose adsorption and 10 mg of MCN was used for cellobiose adsorption). The tubes were capped and vortexed at 25° C. for a period of 24 h in order to achieve equilibrium. The solid-phase concentration of glucan adsorbed on MCN was calculated via material balance from the measured decrease in liquid-phase sugar concentration as measured via HPLC. Thus, the solution was subsequently filtered, and the adsorbate concentration in the filtrate was analyzed by HPLC using a refractive index detector (RID), and compared with the concentration in the standard solution. HPLC-RID analysis was performed using a Shimadzu HPLC equipped with a Biorad Aminex HPX-87H column at 323K. Samples were eluted with a 0.01 NH₂SO₄ mobile phase at a flowrate of 0.6 mL min⁻¹. Products were identified by comparison of retention times with reference compounds. Quantification of mass concentration was determined by the integrated peak area of glucose or cellobiose using a six-point calibration curve.

Cellulose Hydrolysis.

Shorter glucans were synthesized from poly-β-glucans in cellulose and Avicel PH101 (11365), purchased from Fluka Analytical. This protocol involved first dispersing 30 mg of cellulose (Avicel or ¹³C-labeled bacterial cellulose) in 10 mL of concentrated hydrochloric acid (37 wt % aqueous) at room temperature for a period of 1 min. This was followed by the addition of 20 mL of cold concentrated hydrochloric acid (−20° C.), so as to reach a total volume of 30 mL Complete dissolution was achieved at −20° C. after 15 min, and, afterwards, the solution was warmed to 24±1° C. using a water bath for further glucan hydrolysis, during a period of 2 h. During this time, shorter-chain glucans were synthesized from poly-β-glucans originally comprising the cellulose. This timeframe was chosen on the basis of synthesizing a high yield of oligosaccharides relative to glucose monomer. During the final 10 min, MCN was added and adsorption is allowed to occur, under the conditions described below.

Adsorption of Cellotriose, Cellotetraose, and Long-Chain Glucans on MCN.

Standard cellotriose and cellotetraose solutions were prepared in aqueous solution at varying concentrations (10 g L⁻¹, 5 g L⁻¹, 2.5 g L⁻¹, 1.6 g L⁻¹), and 4 g L⁻¹ for cellopentaose. The adsorption isotherms were measured after equilibration using a static method. Preweighed amounts of MCN were placed in 1.5 mL Eppendorf tubes with 0.5 mL of sugar solution (2 mg of MCN was used for all the adsorption). The tubes were capped and equilibrated via vortex mixing at 25° C. for a period of 30 min. The adsorbed glucan concentration on MCN was calculated via material balance from the measured decrease in liquid-phase sugar concentration, as measured via HPLC. Thus, the solution was subsequently filtered, and the adsorbate concentration in the filtrate was analyzed using the Dionex HPLC system described below, and compared with the concentration in the standard solution. For investigating the adsorption of long-chain glucans, preweighed MCN material was placed in a suitable container with predetermined volumes of concentrated acid glucan hydrolysate, as described above, and the resulting slurry was vortexed for 10 min at 4° C. Afterwards, a Speedisk Column (J. T. Baker 8163-04, silica base) was employed for separation of solid MCN via filtration, and the filtered MCN after adsorption was subsequently washed with 3 mL of water in order to remove trace concentrated hydrochloric acid. In order to quantify glucose equivalent content in solution following adsorption, which was in turn used for completing material balances of adsorbed glucan on the basis of glucose equivalents, all glucans in the filtrate solution were hydrolyzed to glucose via concentrated acid hydrolysis. This was accomplished by allowing the collected filtrate to further hydrolyze for 46 h at room temperature, which yielded selectively glucose as the only HPLC-observable product. Only adsorption data representing a significant amount of adsorption via material balance (consisting of differences of at least 10% between standard solution before and filtrate after adsorption) were used in this manuscript. The quantification of oligosaccharides and glucose resulting from glucan hydrolysis was performed via HPLC using a Dionex system (Model ICS-3000), which was composed of: (i) a Dual Pump DP-1, which was used to control the flow rates in the batch chromatography experiments, (ii) an electrochemical detector with dual-detection capabilities configured with gold working electrodes, (iii) an autosampler, (iv) a Carbopac PA-200 analytical column (3×250 mm), and (v) a guard column (3×50 mm) used for oligosaccharide separation. The system was operated by the software Chromeleon Chromatography Management System (version 7.1). The column temperature was maintained at 30° C. The composition of the aqueous sample solution was diluted 200 fold in pure water before analysis with the Dionex HPLC system. Two mobile phases were used to create an eluent gradient, consisting of solution A (0.1 M NaOH) and solution B (0.1 M NaOH+1 M NaOAc) at a constant flow rate (0.4 mL/min) The column was equilibrated at 100% of solution A prior to sample injection. After a 25 μL sample injection, a linear increment of solution B was applied until 13.4% solution B was reached after 25 min. The column was then flushed for 3 min with a 30% solution B composition, before changing the mobile phase composition for 2 min back to 100% solution A, in order to reequilibrate the column before the next sample injection. The NaOH aqueous solution was stored under He. The reagents used for HPLC experiments are used as received and were as follows: D-(+)-cellobiose (99%, Fluka), D-(+)-glucose (99.5%, Sigma), HCl (37%, ACS reagent Sigma), sodium hydroxide solution (50% wt. in water, Fisher). The cellodextrines (cellotriose, cellotetraose, cellopentaose and cellohexose) used in this manuscript were purchased from Seikagaku Biobusiness, Japan in fine grade (>95%). No correction for cellodextrin standard purity was used because the purity is greater than 95%, which was further confirmed via HPLC. Carbon nanopowder (CNP) was purchased from Aldrich (Aldrich #633100), and was treated with aforementioned Soxhlet extraction in water prior to use. Deionized water was obtained from a Milli-Q system by Millipore and was at least 18 MΩ purity. The Langmuir model was employed to analyze measured adsorption isotherms.

Characterization of Adsorbed Glucans on MCN Using MALDI-TOF-MS.

Dry material consisting of MCN-adsorbed oligosaccharide was mixed with 1 μL of a 65 mM DHB (2,5-dihydroxybenzoic acid) in 0.65/0.35 (v/v) acetonitrile/water solution, and this slurry was placed and dried on a stainless-steel sample plate (Shimadzu DE1580TA). MALDI-TOF-MS of this sample was analyzed using a Shimadzu Axima Performance instrument.

Characterization of Adsorbed ¹³C-Labeled Glucans on MCN Using ¹³C DP-MAS NMR Spectroscopy.

¹³C-labeled adsorbed glucan on MCN samples was dried prior to solid-state NMR spectroscopic study (Freeze dry by Labconco Freeze dryer for 12 h under 0.15 mbar). Solid-state ¹³C DP-MAS NMR spectra were obtained using a Bruker DSX-500 spectrometer and a 4 mm Bruker MAS probe. Powder samples (˜15 mg) were packed in a zirconia rotor, and all ¹³C spectra are acquired at a MAS spinning rate of 12 kHz using 90 degree pulses and a recycle delay time of 100 s. All measured chemical shifts were referenced to TMS. In order to quantify the amount of glucose equivalents of ¹³C-labeled glucans on MCN, reference materials consisting of a known amount of ¹³C-labeled glucose on MCN were synthesized as standards. These standard materials were prepared via incipient wetness impregnation using ¹³C-labeled glucose.

Size-Exclusion Chromatography/Gel Permeation Chromatography (SEC/GPC).

Dry adsorbed glucans on MCN material (63.1 mg) were dispersed in 1.9 mL of 0.5% wt LiCl/DMAc, and the slurry was vortexed at room temperature for 14.5 h in order to facilitate glucan desorption into the good solvent. Following glucan desorption, the MCN material was removed via filtration using a Speedisk silica filter, and a second subsequent filtration using a 0.2 μm Teflon syringe filter. SEC/GPC was performed on a Polymer Laboratories PLGPC-50 instrument, equipped with a refractive index concentration detector (RI). Separation was performed on a two-column series consisting of PLGEL-Mesopore 300×7 5 mm preceded by a Mesopore guard column 5 μm particles 50×7.5 mm, Polymer Laboratories. The mobile phase consists of 0.5% wt LiCl in DMAc, and was used at a flow rate of 0.8 mL/min. The oven temperature was set to 50° C. Calibration data were collected for a series of available oligosaccharide standards consisting of glucose, cellobiose, cellotriose, cellotetraose, cellopentaose and cellohexaose. The injection volume is set to 100 μL, and the run time was set to 30 min. Data acquisition and analysis was performed using Cirrus software.

X-Ray Photoelectron Spectroscopy (XPS) Analysis of MCN.

XPS analysis of carbon material was conducted by sprinkling MCN onto double-sided tape using a small spatula. XPS analysis was performed using an Ulvac-Phi Quantera scanning X-ray microprobe operating with a spectral resolution of 1.06 eV. The energy scale of the spectrometer was calibrated using Ag photoemission peaks in accordance with standard practice. XPS results were corrected using the C 1 s peak at 284.6 eV.

Culture Media and Conditions for the Production of ¹³C-Labeled Cellulose.

Gluconacetobacter xylinus (G. xylinus) ATCC 53582 was supplied by American Type Culture Collection (ATCC). The medium culture contained ¹³C-labeled glucose (10 g), peptone (5 g), yeast extract (5 g), disodium phosphate (2.7 g), and citric acid (1.5 g) in one liter of water. A fraction of the aforementioned culture medium (75 mL) was sterilized via filtration, placed in a 100 mL Erlenmeyer flask, and inoculated with a liquid culture of Acetobacter Xylinus. The inoculated media were incubated at 30° C. for 21 days under mild stirring (120 rpm). After incubation, the as-synthesized cellulose in the media was harvested, sliced and washed with water, followed by the reflux with 0.1M NaOH for 15 min, in order to remove buffer and cells from the bacterial cellulose. The purified cellulose was washed with water until neutralization, and then dried for 3 days.

Results and Discussion

Single-Component Adsorption onto MCN.

Glucose and cellobiose adsorption on MCN were performed in order to elucidate the scaling of energetics of adsorption on glucan size. The single-component adsorption isotherm of glucose onto MCN from pH 7 aqueous solution is shown in Table 4 below.

TABLE 4
Glucose adsorption isotherm on MCN material
Glucose Adsorption Isotherm

| | Adsorbed | |
|---|---|---|
| Final | quantity | |
| concentration | (mg/g | |
| (mg/mL) | MCN) | |
| (Ce) | (Qe) | (Ce/Qe) |
| 0 | 0 | 0 |
| 0.44 | 16.5 | 0.026666667 |
| 2.18 | 49.04 | 0.044453507 |
| 6.08 | 88.64 | 0.068592058 |
| 17.14 | 151.49 | 0.113142782 |
| 38.1 | 224.85 | 0.169446298 |
| 38.94 | 213.4 | 0.182474227 |
| 39.1 | 208.97 | 0.187108197 |
| 81.32 | 266.58 | 0.305049141 |
| 83 | 229.75 | 0.361262242 |
| 103.9 | 285.39 | 0.364063212 |
| 105.66 | 250.2 | 0.422302158 |
| 165.7 | 342.71 | 0.483499168 |
| 205.76 | 329.1 | 0.625220298 |

A linear least-squares fit of transformed isotherm data was calculated, and, from this data, Langmuir isotherm parameters related to the binding constant and adsorbent capacity were calculated and summarized in Table 5 below.

TABLE 5
Langmuir constants of glucose at pH 7, cellobiose at both pH 7 and 0,
cellotriose and cellotetraose at pH 7

| | Langmuir Constants* | | |
|---|---|---|---|
| Adsorbates | qₘ (mg · g⁻¹) | b (L · g⁻¹) | R² |
| Glucose | 357 | 0.044 | 0.968 |
| Cellobiose | 556 | 0.31 | 0.996 |
| Cellobiose (pH 0) | 500 | 0.25 | 0.989 |
| Cellotriose | 556 | 4.5 | 0.999 |
| Cellotetraose | 667 | 7.5 | 0.996 |

*qₘ (mg · g⁻¹) and b (L · g⁻¹) are the Langmuir constants, representing the maximum adsorption capacity for the solid-phase loading and the energy constant related to the heat of adsorption, respectively

The measured glucose adsorption capacity of 357 mg glu/g and the limiting slope of the isotherm in the dilute concentration regime of 15.5 mg L/g² were observed to be significantly higher than that reported for activated carbon (corresponding to a capacity of 200 mg glu/g and slope of 2.5 mg L/g²).

The binding constant for cellobiose relative to glucose was investigated in order to determine the effect of incrementally increasing glucan size from monomer to dimer. The adsorption isotherm data of cellobiose onto MCN from both pH 7 and pH 0 aqueous solution are shown in Table 6A below, and Langmuir isotherm parameters are summarized in Table 5 above.

TABLE 6A
Adsorption isotherm data of cellobiose on MCN at pH 7 and pH 0

| | | |
|---|---|---|
| Final | Adsorbed | |
| concentration | quantity | |
| (mg/mL) | (mg/g MCN) | |
| (Ce) | (Qe) | (Ce/Qe) |

Cellobiose Adsorption Isotherm (pH 7)

| | | |
|---|---|---|
| 0 | 0 | 0 |
| 0 | 146.19 | 0 |
| 1.28 | 272.35 | 0.004699835 |
| 1.32 | 279.39 | 0.004724579 |
| 1.4 | 268.82 | 0.005707946 |
| 11.58 | 410.38 | 0.028217749 |
| 13.08 | 382.35 | 0.034209494 |
| 13.16 | 399.59 | 0.032933757 |
| 45.68 | 503.51 | 0.090723124 |
| 82.82 | 511.14 | 0.162029972 |
| 102.28 | 550.71 | 0.185723834 |

Cellobiose Adsorption Isotherm (pH 0)

| | | |
|---|---|---|
| 0 | 0 | 0 |
| 2.08 | 252.3 | 0.008244154 |
| 2.08 | 260 | 0.008 |
| 2.16 | 249.9 | 0.008643457 |
| 13.06 | 360.29 | 0.036248578 |
| 13.64 | 357.33 | 0.038171998 |
| 14.12 | 399.71 | 0.035375611 |
| 46.32 | 425.61 | 0.108832029 |
| 47.54 | 431.75 | 0.110110017 |
| 87.32 | 468.68 | 0.186310489 |
| 88.72 | 441.55 | 0.200928547 |
| 100.62 | 520.38 | 0.193358699 |

The binding constant as represented by the qₘb value for cellobiose was 11-fold higher relative to the glucose value for adsorption onto MCN at pH 7. This scaling of the energetics of adsorption on glucan length for MCN adsorbent is fundamentally different from that previously observed for cation exchange resin adsorbents. The latter materials show a much weaker adsorption (i.e., qₘb values of 0.3-0.8 mg L/g² for glucose) and lack of stronger affinity for the dimer (sucrose) versus monomer (glucose).

Additionally, the scaling of the adsorption coefficient as represented by Langmuir parameter qₘb on the glucan length beyond glucose and cellobiose was also investigated. For this reason, the adsorption isotherms of cellotriose and cellotetraose on MCN material were also measured, under similar conditions as glucose and cellobiose described above (see Tables 6B and 6C below for isotherm data). The Langmuir isotherm parameters corresponding to these isotherms are summarized in Table 5 above.

TABLE 6B
Adsorption isotherm data of cellotetraose

| Final concentration | Adsorbed quantity | |
|---|---|---|
| (mg/mL) | (mg/g MCN) | |
| (Ce) | (Qe) | (Ce/Qe) |
| 0 | 0 | 0 |
| 0.027 | 144.15 | 0.000187305 |
| 0.039 | 397.75 | 9.80515E−05 |
| 0.5 | 514.2 | 0.000972384 |
| 2.37 | 573.81 | 0.004130287 |
| 5.92 | 669.04 | 0.008848499 |

TABLE 6C
Adsorption isotherm data of cellotriose

| Final concentration | Adsorbed quantity | |
|---|---|---|
| mg/mL) | mg/g MCN) | |
| (Ce) | (Qe) | (Ce/Qe) |
| 0 | 0 | 0 |
| 0.04 | 156.8 | 0.000255102 |
| 0.358 | 377.3 | 0.000948847 |
| 0.8 | 390.2 | 0.002050231 |
| 2.11 | 477.5 | 0.004418848 |
| 7.73 | 545.24 | 0.014177243 |

Based on this data, the change in free energy of adsorption between cellobiose and glucose was −1.4 kcal/mol. This change in free energy of adsorption is calculated to be −1.6 kcal/mol between cellotriose and cellobiose, and −0.4 kcal/mol between cellotetraose and cellotriose. Altogether, this data demonstrates that the free energy of adsorption monotonically decreases as the glucan chain length increases from glucose to cellotetraose, and this energy decreases nearly uniformly when going from glucose to cellobiose, and when going from cellobiose to cellotriose. Specifically, a lower decrease in free energy of adsorption was observed when going from cellotriose to cellotetraose. Although adsorption phenomena may depend on many variables such as solvation, they are also highly dependent on the degree of contact on the molecular level, between the curved carbonaceous MCN material surface and the short-chain glucans above. Short-chain glucans are typically rigid, whereas, on a certain length scale, the MCN surface is typically curved. On this length scale, the results of this example suggest that good contact between the curved MCN surface and rigid short-chain glucans has been prevented due to incompatible geometries (curved MCN surface and rigid glucan have less overlap). The data in this example suggest curviness on the length scale of a cellotetraose molecule in the MCN material on the molecular level, given the lower drop in free energy of adsorption when going from cellotriose to cellotetraose.

Additionally, the data in this example demonstrate that the binding coefficient continues to increase with glucose chain length. Based on the known footprint for a cellobiose repeat unit in crystalline cellulose (0.81 nm²/cellobiose)²², and the MCN material BET surface area, an upper bound for the maximum theoretical coverage of adsorbed cellobiose on the MCN material is calculated to be 1269 mg cellobiose/g MCN. The measured saturation surface concentration for cellobiose adsorption was calculated from data in Table 6A above to be 556 mg cellobiose/g MCN. This value is less than half of the maximum theoretical value and indicates that the distance between adsorbed cellobiose units on MCN is on average roughly 1.5-fold higher than this distance in crystalline cellulose.

From the perspective of applying carbon materials directly to an acid-containing hydrolysate stream without the need for neutralization, cellobiose adsorption at low pH was investigated. Data in Tables 5 and 6 above show that at a lower pH of 0, there is only a slight decrease in qₘb (1.37-fold lower at low pH) and qₘ (10.0% lower at low pH) relative to values at neutral pH, suggesting that carbon can be used as a versatile adsorbent for glucans equally well at low and neutral pH.

Cellopentaose was chosen as a compound that should fit inside of the MCN interior porosity (radius of gyration is predicted to be 0.76 nm, which is significantly smaller than the MCN pore diameter of 3.2 nm). The timescale for adsorption of this oligosaccharide is depicted in Table 7 below.

TABLE 7
Mass of cellopentaose adsorbed on
MCN as a function of adsorption time

| | |
|---|---|
| | [G5]/MCN |
| Time of adsorption | mgG5/gMCN |

| min | Stdev | avg |
|---|---|---|
| 1.5 | 18 | 414 |
| 5.5 | 2 | 471 |
| 15.5 | 90 | 501 |
| 30.5 | 12 | 381 |

The kinetics of adsorption when treating 0.3 mL of a 4 g/L cellopentaose solution with 2 mg of MCN material was monitored. Even after 1 min, adsorption is equilibrated, at the level of 50% of the cellopentaose in solution adsorbed in this case. The high adsorbed coverage of cellopentaose was also observed to be 400 mg adsorbed cellopentaose/g carbon.

Multi-Component Adsorption onto MCN.

Multi-component adsorption studies using MCN were accomplished by performing a brief (2 h) concentrated acid (37% aqueous HCl at room temperature) treatment of Avicel crystalline cellulose for selective depolymerization. This enabled the synthesis of a glucan solution (glucan content is approximately 1000 mg of glucose equivalents per L) that had varying hydrolyzed chain lengths.

XPS analysis of MCN before and after treatment with concentrated HCl under typical conditions (as described below) was performed. Results demonstrate lack of change to the carbon species present within the MCN material. These species include hydrocarbons, alcohol/ether, carbonyl, and carboxyl functionalities. A very slight incorporation of chloride (<0.1 atom %) was observed as a result of the concentrated HCl treatment. These minor effects do not appear to have significantly changed the adsorption characteristics of the MCN material.

Brief treatment (10 min) of this concentrated acid hydrolysate solution with MCN was observed to cause glucan adsorption from solution. Such an approach potentially offers a method of recycling acid used during concentrated acid cellulose depolymerization, by removing depolymerized sugar fragments via adsorption onto MCN and thereby permitting reuse of the concentrated acid stream. This method can be used in conjunction with the concentrated acid process for the dissolution and hydrolysis of cellulose.

The rapid kinetics of long-chain glucan adsorption onto MCN was demonstrated in the kinetic data shown in Table 8 below.

TABLE 8
Mass of glucans adsorbed on MCN as a function of adsorption
time for 10 ml glucan solution volume treated with on 20 mg MCN

| | average | st dev |
|---|---|---|
| Time | mg_{Glu} | mg_{Glu} |
| min | eq./g_{CM} | eq./g_{CM} |
| 2.5 | 194.78 | |
| 6.5 | 186.99 | 41.87 |
| 11.5 | 205.60 | 24.51 |
| 31.5 | 208.60 | 19.70 |
| 61.5 | 176.04 | |

These data were determined by measuring the difference in concentration of glucose equivalents (measured by fully hydrolyzing glucan solution in concentrated acid) for solutions before and after MCN treatment. The data showed that the minimum equilibration time investigated of 4 minutes (limited by filtration time to separate filtrate solution from MCN) was sufficient for glucan adsorption. This dynamic study of adsorption of glucans from cellulose solution was performed in a concentrated regime, consisting of adsorption of long-chain glucans from the concentrated acid hydrolysate. The observed rapid adsorption time was unexpected given the large sizes of the glucans adsorbing (relative to the uniform mesopore opening in MCN).

In all subsequent multi-component adsorption experiments described in this example, a nominal period of 10 minute equilibration time was used, which helped to minimize further glucan hydrolysis during treatment with MCN, which would otherwise occur at longer equilibration times. Two extreme adsorbed concentration regimes were studied as follows: (1) uptake of glucan on MCN in a regime where there was excess glucan relative to binding sites on MCN, which resulted in a highly concentrated adsorption regime; and (2) uptake of glucan on MCN in a regime where there was excess binding sites on MCN relative to glucan in hydrolysate solution, which resulted in a highly dilute adsorption regime. Dilute and concentrated within this context refer to relative amounts of adsorbed glucan concentrations on the MCN surface, and specifically do not refer to solution-phase concentrations. Within this context, concentrated represented amounts of adsorbed glucose equivalents that are higher than 200 mg glucose equivalents per gram of MCN.

The dilute and concentrated regimes of glucan adsorption onto MCN material were investigated in experiments (A)-(D) (dilute) and (E)-(H) (concentrated) in Table 9 below and FIG. 18.

TABLE 9
Concentration of adsorbed glucans on MCN

| | | [Glucan] | [Glucan] | [Glucan] after | | | | |
|---|---|---|---|---|---|---|---|---|
| | [Glucan] | before | after | adsorption + | | | | |
| | Control | adsorption | adsorption | 46 h | Percentage of | | Mass of | |
| | experiment | (G1-G6) | (G1-G6) | hydrolysis | Adsorbed | Solution | MCN | [Glucan] on |
| | mg_{Glu eq.}/L | mg_{Glu eq.}/L | mg_{Glu eq.}/L | mg_{Glu eq.}/L | Glucose | volume | engaged | MCN |
| Exp. | Stage (I) | Stage (II) | Stage (III) | Stage (IV) | Equivalents (%) | (mL) | (mg) | mg_{Glu eq.}/g_{MCN} |
| (A) | 1030 | 520 | 204 | 204 | 75% | 1.0 | 40.1 | 19.3 |
| (B)* | 952 | 443 | 158 | 156 | 80% | 1.0 | 39.8 | 19.0 |
| (C) | 1030 | 520 | 290 | 300 | 66% | 1.0 | 19.4 | 34.8 |
| (D)* | 952 | 443 | 218 | 220 | 73% | 1.0 | 20.0 | 34.6 |
| (E) | 992 | 415 | 446 | 556 | 42% | 10.0 | 20.0 | 209 |
| (F) | 992 | 415 | 465 | 707 | 28% | 20.1 | 20.0 | 282 |
| (G) | 992 | 415 | 478 | 833 | 15% | 39.4 | 19.8 | 303 |
| (H)* | 711 | — | — | 572 | 19% | 39.3 | 20.4 | 262 |
| (I) | 919 | — | — | 674 | 27% | 10.0 | 311.2 | 7.7 |

[Glucan] = Concentration of poly-β-glucans
*¹³C cellulose experiments

In all of these experiments, the initial concentration of glucose equivalents in the concentrated acid hydrolysates (i.e., stage (I) in FIG. 18) is similar. After treatment of a given volume of the stage (II) solution with the indicated amount of MCN (see Table 9 above for MCN mass and solution volume), a stage (III) stream is produced in FIG. 18 after MCN removal via filtration.

HPLC was used to quantify soluble oligosaccharides ranging from glucose (G1) up to cellohexaose (G6) due to availability of calibration standards. Using HPLC, the concentration of glucose equivalents present in species G1-G6 within the stage (III) stream was measured, and this HPLC data was presented in Table 9 above. In order to ascertain the total glucose content of the filtrate solution at stage (III), the stage (III) solution was allowed to fully hydrolyze to glucose during a period of 46 h at room temperature, thereby synthesizing a stage (IV) glucose solution in FIG. 18. Control experiments demonstrate that the selectivity of this latter hydrolysis process was nearly 100% (i.e. correspondence between amount of Avicel added per unit volume (1 g Avicel/L) for synthesis of standard solution for adsorption and stage (I) column in Table 5 above). Therefore, the difference in glucose equivalents between solutions at stages (I) and (IV) in FIG. 18 represents the total amount of glucose equivalents adsorbed onto MCN for each experiment. The aforementioned experiments represent a material balance on the control system labeled as dashed line a in FIG. 18.

Adsorbed glucan coverages of up to 303 glucose equivalents per gram of MCN were achieved (see experiment (G) in Table 9 above). The fraction of initial glucose equivalents available in Stage (I) that were adsorbed varies from 66%-80% in dilute experiments (A)-(D) and from 15%-42% in concentrated experiments (E)-(H) in Table 9 above. The measured concentration of glucose equivalents in species G1-G6 in the stage (III) stream (i.e. in the filtrate following adsorption onto MCN) and the total glucose equivalents at the fully hydrolyzed stage (IV) stream were virtually identical for all dilute regime experiments (A)-(D) in Table 5. This result suggests that the MCN adsorbed all fragments larger than G6 that were present in the original glucan hydrolysate solution for the dilute experiments (stage (II)).

Table 10 below summarizes data that demonstrates the increasing favorability of adsorbing longer-chain glucans for experiment (C) using HPLC data of glucose and oligosaccharides G2-G6. The difference between the data before and after adsorption for a particular glucan represents the amount of that glucan that has been adsorbed onto the MCN. It is evident from the data in Table 10 that almost all G6 was adsorbed, whereas systematically less G5, G4, G3, cellobiose (G2), and glucose (G1) were adsorbed.

TABLE 10
Distribution of glucose to cellohexaose before and
after the adsorption by MCN in a dilute regime

| | Concentrations (mg Glu/L) | | | | | |
|---|---|---|---|---|---|---|
| | Glc | Ceb | G3 | G4 | G5 | G6 |
| Before | 102.18 | 65.65 | 85.33454 | 90.79 | 89.85 | 86.32 |
| After | 86.95 | 52.72 | 59.15867 | 45.80 | 29.17 | 16.27 |

Altogether, the results in Table 10 show that all glucans longer than cellohexaose (G6) were adsorbed by the MCN in the dilute regime, due to their preferential adsorption energetics. The physical origin for this selectivity could be the increasing favorability of adsorption for longer-chain glucans, as shown by the monotonically decreasing free energy of adsorption for the series glucose to cellotetraose on MCN, as described above. The total amount of G1-G6 glucan adsorbed in experiment (C) was 12.1 mg of glucose equivalents per gram of MCN material, which is 65% smaller than the total glucan adsorption in this experiment. In the concentrated adsorption regime corresponding to experiments (E)-(H), saturation of the carbon surface with adsorbed glucan limits the fractional recovery of glucans larger than G6 to be below 50%.

To close the material balance represented in Table 9 above, adsorption was quantified via direct interrogation of bound species on the MCN material. ¹³C-labelled cellulose was used as a cellulose source in the adsorption experiments described above in order to close the material balance via characterization of the solid, which was synthesized by using bacteria and ¹³C-labeled glucose. Adsorption was performed in the dilute regime using ¹³C-labelled cellulose according to similar methods described above, to synthesize MCN materials consisting of adsorbed glucan that are equivalent to materials resulting from experiments (B) and (D) in Table 9 above. In addition, a sample was synthesized by performing adsorption in the concentrated regime using ¹³C-labelled cellulose. This sample consisted of MCN containing an adsorbed glucan coverage that was similar to experiment (H) in Table 9 above. All of the above ¹³C-labelled adsorbed glucan on MCN materials were investigated via Bloch Decay solid-state ¹³C NMR spectroscopy. The corresponding spectra were shown in FIG. 16. Also shown in FIG. 16(a) is an assigned spectrum for neat ¹³C-labeled bacterial cellulose (not adsorbed on support). The spectra of adsorbed glucan on MCN in FIGS. 16(b) and 16(c) both lack the sharp characteristic resonances associated with crystalline cellulose for carbons labeled 4 and 6 in FIG. 16, and, instead, show merging of resonances associated with these carbons together with resonances of carbons 2, 3, and 5 in FIG. 16. In addition, spectra of adsorbed glucans in FIG. 16 exhibit characteristic amorphous resonances, which are shifted slightly upfield relative to their crystalline counterparts (i.e. upfield-shifted shoulder at ˜62 ppm). These amorphous resonances are clearly visible as minor contributors in the crystalline NMR spectrum of FIG. 16(a) at ˜62 ppm and ˜85 ppm. The observed broad resonance centered at around 120 ppm in FIGS. 16(b) and 16(c) was attributable to aromatic groups consisting of the MCN support.

A calibration curve for ¹³C Bloch Decay integrated intensity in the region between 40 ppm and 110 ppm versus amount of adsorbed ¹³C-labeled glucose equivalents was used to quantify amount of adsorbed glucan. Such a calibration curve was prepared by impregnating known amounts of ¹³C-labeled glucose monomer onto a known amount of MCN support. Table 11A summarizes data for the calibration curve. Table 11B below summarizes the comparison of amount of adsorbed glucose equivalents as measured via ¹³C Bloch Decay NMR spectroscopy from data in FIG. 16 and HPLC data in Table 9 above.

TABLE 11A
Calibration curve data

| Mass 13C glucose/ | |
|---|---|
| Mass TKTMS(13C fraction from | Area Glucose/ |
| natural abundance) | area(TKTMS) |
| Calibration: | |
| 122.02 | 90.80 |
| 44.17 | 35.06 |
| 19.51 | 16.24 |
| 11.19 | 10.64 |
| 7.55 | 6.42 |
| slope | 0.75 |
| R2 | 0.9975 |
| Measure: | |
| 89.43 | 67.39 |
| 11.53 | 8.69 |
| 9.52 | 7.18 |

TABLE 11B
Measurement Comparison Between HPLC
and ¹³C DP-MAS NMR Spectroscopy

| | | Concentration of |
|---|---|---|
| | Concentration of | Adsorbed ¹³C |
| | Adsorbed ¹³C | Glucans on MCN |
| | Glucans on MCN | (mg of glucose |
| | (mg of glucose | equivalent/g of |
| | equivalent/g of | MCN) by ¹³C DP- |
| | MCN) by HPLC | MAS Solid-state |
| Samples | Analysis | NMR |
| (B)* | 19 | 20 |
| (D)* | 34.6 | 38 |
| (H)* | 262 | 258 |

The spectrum in FIG. 16(b) corresponds to 20 mg of glucose equivalent per gram of MCN (corresponds to experiment (B) in Table 9) whereas the spectrum in FIG. 16(c) corresponds to 258 mg of glucose equivalent per gram of MCN (corresponds to experiment (H) in Table 9). These data represent the extremes of low and high loadings of glucan on MCN, and show excellent agreement for experiments (B), (D), and (H) in Table 9 above between ¹³C Bloch decay NMR spectroscopy measured values and those determined via HPLC. This data indicates closure of the material balance of data in Table 9 above.

GPC was used to further gain further insight into the DP (degree of polymerization) distribution of adsorbed glucan species on the MCN support. This was accomplished by washing the MCN following adsorption with a good glucan solvent such as 0.5% wt LiCl/DMAc, and analyzing the filtrate via GPC. Quantitative analysis of GPC data was enabled by using calibration standards consisting of G1-G6. Response coefficients for these short glucans were shown in Table 12 below, and were observed to be concentration independent.

TABLE 12
GPC concentration response coefficient as a function
of the number of glucose units in oligosaccharide
standards for different dilution factors: no dilution, 1:5, 1:10

| Oligosaccharide | Concentration response (mg mL⁻¹ mV⁻¹) | | |
|---|---|---|---|
| Length (Glucose | no dilution | | |
| units) | (1:1) | 1:5 | 1:10 |
| 1 (glucose) | 1.65 × 10⁻⁴ | 1.62 × 10⁻⁴ | 1.67 × 10⁻⁴ |
| 2 (cellobiose) | 1.78 × 10⁻⁴ | 1.79 × 10⁻⁴ | 1.80 × 10⁻⁴ |
| 3 (cellotriose) | 1.93 × 10⁻⁴ | 1.91 × 10⁻⁴ | 1.95 × 10⁻⁴ |
| 4 (cellotetraose) | 2.04 × 10⁻⁴ | 2.03 × 10⁻⁴ | 2.06 × 10⁻⁴ |
| 5 (cellopentaose) | 1.99 × 10⁻⁴ | 2.00 × 10⁻⁴ | 2.02 × 10⁻⁴ |
| 6 (cellohexose) | 2.02 × 10⁻⁴ | 2.05 × 10⁻⁴ | 2.09 × 10⁻⁴ |

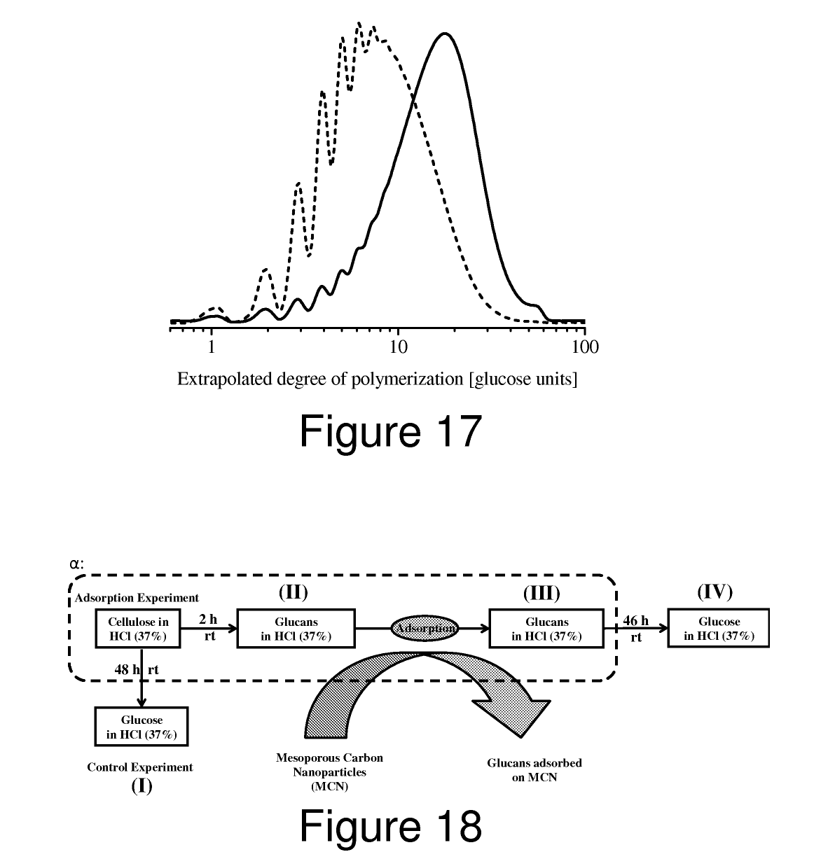

The observed constancy of the response factors for cellotetraose (G4), cellopentaose (G5), and cellohexaose (G6) in Table 12 above suggests that higher oligosaccharides and glucans may also have the same response factor as these last three species. Based on this assumption and the measured response factors for G1-G6, FIG. 17 represents the distribution of glucans removed from the washing procedure from the MCN. The dashed line in FIG. 17 corresponds to the GPC analysis of a representative dilute-regime experiment that is similar to (C) in Table 9 above, whereas the solid line in FIG. 17 corresponds to the GPC analysis of concentrated-regime experiment (G).

Calibrated quantification of the GPC data in FIG. 17 was shown in Table 13.

TABLE 13
Oligosaccharide/glucan concentration of GPC analysis

| Degree of | Concentration on | Concentration on |
|---|---|---|
| polymerization | carbon [mg/g_{MCN}] | carbon [mg/g_{MCN}] |
| [glucose units] | Lower Loading | Higher Loading |
| 1 | 0.37 | 0.51 |
| 2 | 1.16 | 1.28 |
| 3 | 2.80 | 2.33 |
| 4-6 | 15.42 | 15.95 |
| 7-10 | 12.80 | 32.63 |
| 11-40+ | 13.49 | 149.48 |
| Total [mg/g_{MCN}] | 46.04 | 202 |

For the dilute-regime experiment, the total amount of glucose equivalents/g adsorbed on the MCN material as determined via GPC corresponds well with that measured by HPLC. This supports the extrapolation in the GPC response coefficient for glucans larger than cellohexaose, and further reinforces the closure of the material balance in Table 9 above. The fraction of glucans adsorbed on the MCN that fall within the DP range corresponding to G1-G6 is 43%. Again this supports the assertion that simply monitoring adsorption onto MCN using HPLC of G1-G6 fragments inevitably leads to incomplete data, since a significant fraction (i.e., 57% in this case) of mass adsorbed arises from species that are larger than G6, and therefore cannot be reliably traced using HPLC analysis of soluble glucans. This holds even for the dilute regime of adsorption on MCN, where this effect is minimized relative to concentrated adsorption of glucan on MCN. The slight mismatch in fraction of adsorbed species larger than G6 between GPC and HPLC data is likely due to errors in deconvoluting the distribution of various species from the GPC data in FIG. 17. For the concentrated-regime experiment, the total amount of glucose equivalents/g adsorbed on the MCN as determined via GPC is only 67% of that measured using HPLC for experiment (G) in Table 9 above. This discrepancy is explained by the inability to extract the remaining ˜33% of glucans adsorbed on carbon using the washing procedure employed here. Such a result is consistent with the shifted distribution of the GPC data in FIG. 17 towards higher DP for the concentrated-regime experiment, and is likely due to a greater availability of long chains that are present in excess during the adsorption experiment, as a result of the larger glucan hydrolysate solution volume to MCN weight ratio used during adsorption. Therefore, as a result of this incomplete extraction of high DP glucans from the concentrated-regime experiment, only a lower-bound estimate for the fraction of adsorbed glucans with DP greater than G6 can be obtained from data in Table 13 above. This fraction corresponds to 90% of the total adsorbed glucans in the concentrated regime experiment, and was further increased to 93% when using the total adsorbed glucans from HPLC data (reasonably assuming that the unextracted glucans have a higher DP than cellohexaose).

Using data in Table 13 above, glucans having a DP higher than 10 accounted for about 30% of the adsorption for the dilute-regime experiment and at least 74% of the adsorption for the concentrated regime experiment G. The first direct proof of adsorbed glucans larger than a DP of 10 was provided by matrix-assisted laser desorption/ionization mass spectrometry (MALDI-MS) The MCN used as a support for the adsorbed glucans functions was an efficient matrix for the MALDI-MS experiment. MALDI-TOF-MS spectra were shown in FIG. 14 for dilute-regime experiments using both ¹³C-labeled and unlabeled glucan.

TABLE 14
MALDI-TOF-MS spectra data of adsorbed oligosaccharides/
glucan and ¹³C labeled oligosaccharides on MCN

| Oligomer | Regular | ¹³C glucans |
|---|---|---|
| of interest | glucans (m/z) | (m/z) |
| 3 | 527 | 559 |
| 4 | 689 | 727 |
| 5 | 851 | 895 |
| 6 | 1013 | 1063 |
| 7 | 1175 | 1225 |
| 8 | 1337 | 1393 |
| 9 | 1499 | 1561 |
| 10 | 1661 | 1729 |
| 11 | 1823 | 1897 |
| 12 | 1985 | 2065 |
| 13 | 2147 | 2233 |
| 14 | 2309 | 2401 |
| 15 | 2471 | 2569 |
| 16 | 2633 | 2737 |

Even for these experiments, where the amount of high DP glucans is expected to be less than for the concentrated-regime experiments, the MALDI-TOF-MS data in Table 14 above demonstrate adsorbed fragments consisting of DP higher than G10. The molecular peaks in the MALDI-TOF-MS spectrum correspond to the complexation of oligosaccharides with ions after laser bombardment, which results in the formation of [mass+Na]⁺ molecular species. The interval mass number of 162 (m/z) for the unlabeled glucan (168 for the ¹³C-labeled glucan) represents the dehydrated glucose monomer (—C₆H₁₀O₅)ₙ—) in the adsorbed glucan. The inset data clearly show adsorbed fragments up to and including DP of 16.

Thus, glucans with a radius of gyration larger than the pore radius of the MCN were observed to enter and adsorb on the MCN surface, supporting the high adsorbed amounts shown in Tables 9 and 13 above. For the MCN materials used here, this limit corresponds to a DP of 10 glucose repeat units. Without wishing to be bound by any theory, a possible answer is that adsorption occurs on the interior surface area of the MCN, facilitated by change in glucan phase conformation once adsorbed on the surface, which allows the glucan strands to diffuse into the interior porosity of the MCN.

Additionally, to determine whether glucan chain adsorption occurs on internal surface within the MCN mesopores, the following control experiment was conducted. Both MCN and a commercially available (Aldrich #633100) graphite-type carbon consisting of 50 nm particles (CNP) were equilibrated with concentrated acid glucan hydrolysate. The details of this treatment correspond to experiment (I) in Table 9 above. Thus treatment of a 10 mL hydrolysate solution with 311 mg of CNP material resulted in 27% of glucose equivalents within the hydroylzate being adsorbed. This corresponds to a maximum adsorbed glucan loading of 7.7 mg glucose equivalents adsorbed per gram of CNP materials. Under virtually identical conditions, MCN adsorbed up to 27-fold more glucans per gram, as shown by experiments (E)-(H) in Table 9 above. However, because the CNP has higher external surface area per gram (78.5 m²/g) relative to MCN (55.5 m²/g), this data unequivocally demonstrates a lack of correlation between external surface area and glucan adsorption capacity. Therefore, this result strongly suggests that mesopores are important, and the active sites are responsible for glucan adsorption in MCN materials.

Conclusions

The results from this example demonstrate that MCN materials can adsorb glucans from concentrated acid hydrolysate in amounts of up to 30% by mass, in a manner that causes preferential adsorption of longer-chain glucans. Greater absolute free energy of adsorption per additional glucose unit within the chain, for glucans with a higher DP was observed, based on the monotonically decreasing free energy of adsorption for the series glucose to cellotetraose on MCN. Within this series, the free energy of adsorption decreases by at least 0.4 kcal/mol for each glucose repeat unit within the chain. On the other hand, a graphite-type carbon nanopowder (CNP), which lacks internal mesoporosity, was only capable of adsorbing glucans from concentrated acid hydrolysate in an amount less than 10% by mass (7.7 mg/g of CNP), despite having a higher external surface area relative to MCN material. The inefficiency of CNP adsorption can be attributed to the lack of internal mesoporosity. HPLC of hydrolyzed fragments in solution, ¹³C Bloch Decay NMR spectroscopy, and GPC give provide good agreement in terms of adsorbed glucan coverage on MCN. The latter and MALDI-TOF-MS provided evidence for adsorption of large glucans on the MCN surface, which have a radius of gyration larger than the pore radius of the MCN material. Material balances based on HPLC data also require the adsorption of such large species.

### Example 15

### Glucose Release from an Adsorbed Glucan on MCN at Room Temperature

This example demonstrates that the glucose content present in glucans that are strongly adsorbed to the surface of MCN can readily be recovered in the form of glucose in solution via the following treatment. Adsorbed long chain glucan on the MCN surface were treated with 37% HCl at room temperature. This treatment causes hydrolysis of adsorbed long chain glucan to glucose, which was recovered in greater than 95% yield relative to glucose equivalents originally present in the adsorbed long chain glucan.

Materials and Methods

Glucan Adsorption.

Adsorbed glucan on MCN was synthesized by treating (i) 10 mL of a mixture consisting of Avicel cellulose (2gGlu.eq./L) in aqueous HCL (37%) for 2 h, with (ii) 40 mg of MCN, for 10 min at room temperature, to yield a loading of 150 mg Glucose equivalent/g of material.

Glucan Desorption.

25 mg of MCN containing adsorbed long chain glucans, was treated with 1.5 mL concentrated aqueous HCl (37%) at room temperature for 12-64 h under mild stirring. After filtration to remove solids, and a brief wash consisting of (i) 1.5 mL of concentrated aqueous HCl (37%) at room temperature and (ii) 5 mL of hot water wash, recovered glucose filtrate is analyzed in HPLC.

Results

Results of recovery in the filtrate versus time of hydrolysis are shown in Table 15 below. These results demonstrate the recovery, via hydrolysis/desorption of glucose, of more than 95% of the glucose content that was originally present in the adsorbed long-chain glucan on MCN composite material, using the approach described above.

TABLE 15
Kinetic data of glucose release from MCN
containing adsorbed long chain glucan

| | | |
|---|---|---|
| Time | Glucose | G2-G5 |

| hour | AVG | Stdev | % |
|---|---|---|---|
| 14 | 59% | 2.6% | 26% |
| 26.5 | 88% | 3.1% | 10% |
| 39.5 | 97% | 3.3% | 4% |
| 45 | 98% | 2.8% | 1% |
| 64 | 98% | 2.1% | 0% |

### Example 16

### Glucose Release from Glucan Adsorbed HSO₃-MCN (Internal Acid)

This example demonstrates that adsorbed glucans on sulfonated MCN can be released in up to 85% yield as glucose, via treatment with warm water. The glucose content present in glucans that are adsorbed to the surface of HSO₃-MCN can readily be recovered in the form of glucose in solution via the following treatment. Adsorbed long-chain glucans on the MCN-SO₃H surface were treated with water at 150° C. This treatment causes hydrolysis of adsorbed long-chain glucan to glucose, which is recovered in greater than 80% yield relative to glucose equivalents originally present in the adsorbed long-chain glucans.

Materials and Methods

Glucan adsorption on HSO₃-MCN. Adsorbed glucan on HSO₃-MCN was synthesized by treating (i) 10 mL of a mixture consisting of Avicel cellulose (2gGlu.eq./L) in aqueous HCL (37%) for 2 h, with (ii) 40 mg of HSO₃-MCN, for 10 min at room temperature, to yield a loading of 100 mg Glucose equivalent/g of HSO₃-MCN.

Glucose Desorption from HSO₃-MCN.

20 mg of HSO₃-MCN containing adsorbed long-chain glucans was treated with 1 mL of milliQ water, at 150° C. under mild stirring. After filtration the carbon material was briefly washed with 4 mL warm water (80° C.). Recovered glucose filtrate was analyzed by HPLC.

Results

Results of recovery in the filtrate versus time of hydrolysis are shown in Table 16 below. These results demonstrate that it is possible to recover via hydrolysis/desorption of glucose more than 80% of the glucose content that was originally present in the adsorbed long-chain glucan on HSO₃-MCN composite material, using the approach described above.

TABLE 16
Kinetics of glucose release from SO₃H-MCN
containing adsorbed long-chain glucans.

| Time | % of glucose |
|---|---|
| (150° C., water) | recovery |
| 6 | 48% |
| 12 | 74% |
| 24 | 85% |

### Example 17

### Adsorption of Xylan on MCN and Acid-Functionalized MCN

This example demonstrates the (i) release of xylans from raw biomass via pretreatment followed by (ii) treatment of the released xylans, which are present under either acidic, neutral, or basic solution pH, with MCN as adsorbent.

Materials and Methods

1 g of Miscanthus was placed in a microwave canister and added to 10 g of MillQ water. A microwave reactor was programmed to heat up to a designated temperature between 190° C.-200° C. and a specified residence time between 9 min-20 min. After reaction, the canister was cooled in an ice bath, and the solution was filtered using a glass fiber filter. 1 mL of the filtrate was acidified using 0.09 mL of 30 wt % H₂SO₄. The standard NREL hydrolysis protocol was subsequently used to determine the mass of xylan recovered from Miscanthus. See “Determination of Sugars, Byproducts, and Degradation Products in Liquid Fraction Process Samples”, Laboratory Analytical Procedure (LAP), Issue Date: Dec. 8, 2006, A. Sluiter, B. Hames, R. Ruiz, C. Scarlata, J. Sluiter, and D. Templeton. Results are summarized in Table 17 below.

TABLE 17
Removed hemicellulose from Miscanthus

| | | | | | | Removed | | Percentage of |
|---|---|---|---|---|---|---|---|---|
| | | | | | | Percentage of | | total |
| | | | | | | total | | hemicellulose |
| | Experimental | Glucan | Xylan | Arabinan | Hemicelluose | hemicellulose | Ratio | removed as |
| Samples | Conditions | (mg) | (mg) | (mg) | (mg) | (%) | (Xylan/Xylose) | xylan (%) |
| (A) | 190° C., 20 min | 12.2 | 126 | 11.0 | 137.0 | 61.5 | 3.5 | 71 |
| | (1 g/10 mL) | | | | | | | |
| (B) | 190° C., 10 min | 34.5 | 273.6 | 31.2 | 304.8 | 45.9 | 9.2 | 81 |
| | (large scale, | | | | | | | |
| | 3 g/30 mL) | | | | | | | |
| (C) | 200° C., 9 min | 11.9 | 124.0 | 9.4 | 133.4 | 60.2 | 4 | 74 |
| | (1 g/10 mL) | | | | | | | |
| (D) | 200° C., 10 min | 12.3 | 128.7 | 10.6 | 139.3 | 62.9 | 3.1 | 70 |
| | (1 g/10 mL) | | | | | | | |

The optimal condition in Table 17 above included removing approximately 46% hemicellulose content of Miscanthus originally present, with a xylan/xylose ratio of approximately 9.2. This represents 81% of hemicellulose removed as xylan. Maintaining the latter ratio high facilitates a high degree of affinity for xylan adsorption to MCN.

1 mL of sample (B) in Table 17 above was treated with 10-30 mg of MCN material, and 10-40 mg of a sulfonic acid—functionalized MCN material at room temperature for 30 min. Afterward, the amount of adsorbed xylan is quantified by filtering and analyzing the filtrate. The filtrate is analyzed via HPLC by first hydrolyzing xylan in the filtrate to xylose using standard NREL hydrolysis conditions. Use of less MCN adsorbents in the preceding sentence results in a higher adsorption coverage, and results are shown in Table 18 below.

TABLE 18
Adsorption of Xylan on MCN and SO₃-MCN in
different concentration regimes

| | High Adsorbed | Medium Adsorbed | Low Adsorbed |
|---|---|---|---|
| | Xylan Surface | Xylan Surface | Xylan Surface |
| | Coverage | Coverage | Coverage |
| MCN | 564.10 mg/g* | 404.15 mg/g | 310 mg/g |
| | (51%) | (77%) | (88%) |
| HSO₃-MCN | 454.46 mg/g | 351.35 mg/g | 233.67 mg/g |
| | (41%) | (68%) | (88%) |

*Xylose equivalents
Xylose concentration equals 8 wt % after the pretreatment with water at 190° C. for 10 min (large scale, 3 g/30 mL); Xylan/xylose = 12

For MCN, a high adsorbed xylan coverage of 564.1 mg xylose equivalents/g MCN was achieved by treating with 10 mg of MCN, a medium adsorbed xylan coverage of 404.15 mg xylose equivalents/g MCN was achieved by treating with 20 mg of MCN and a low adsorbed xylan coverage of 310 mg xylose equivalents/g MCN was achieved by treating with 30 mg of MCN. For sulfonic acid-functionalized MCN, a high adsorbed xylan coverage of 454.46 mg xylose equivalents/g SO₃-MCN was achieved by treating with 10 mg of SO₃-MCN, a medium adsorbed xylan coverage of 351.35 mg xylose equivalents/g SO₃-MCN was achieved by treating with 20 mg of SO₃-MCN and a low adsorbed xylan coverage of 233.67 mg xylose equivalents/g SO₃-MCN was achieved by treating with 40 mg of SO₃-MCN.

This example demonstrates that MCN can adsorb long-chain xylan in amounts of up to 50% by mass under neutral conditions. Acid-functionalized MCN (such as sulfonic acid-functionalized MCN) performs similarly, as also shown in Table 18 above.

### Example 18

### Hydrolysis of Adsorbed Xylan on MCN and Acid-Functionalized MCN

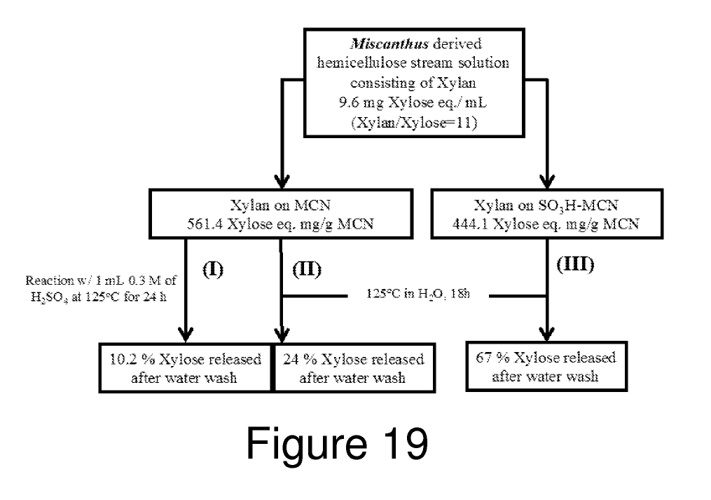

Following adsorption of xylan as described above in Example 17, this example demonstrates adsorbed xylan can be released as soluble xylose in solution. Three possible approaches for accomplishing this in FIG. 19, which are labeled in the figure as (I) dilute sulfuric acid, (II) MCN, and (III) functionalized MCN approaches.

Approach (I) was conducted using 10 mg of high adsorbed xylan coverage MCN with 1 mL of 0.3M H₂SO₄ at 125° C. for 24 h, and this resulted in 10.2% of xylose equivalents that were originally adsorbed being released into solution.

Approach (II) was conducted using 10 mg of high adsorbed xylan coverage MCN with 0.5 mL of MillQ water at 125° C. for 18 h, and this resulted in 24% of xylose equivalents that were originally adsorbed being released into solution.

Approach (III) was conducted using 10 mg of high adsorbed xylan coverage MCN with 0.5 mL of MillQ water at 125° C. for 18 h, and this resulted in 67% of xylose equivalents that were originally adsorbed being released into solution. Results obtained using approach (III) in Scheme 1 showed that a larger fraction of adsorbed xylan can be recovered as soluble xylose.

### Example 19

### Separation of Cellulose (Hexose) and Hemicellulose (Pentose) Streams During Concentrated Mineral Acid Processing of Biomass

This example demonstrates a method for separating a mixture of pentose and hexose sugars that may be produced by the hydrolysis of biomass containing hemicellulose and cellulose.

A. Kinetics of Cellulose Versus Hemicelluloses Hydrolysis Upon Treating Raw Biomass with Concentrated Mineral Acid

80 mg of Miscanthus biomass (0.12 mm particles) were contacted with 13 mL concentrated hydrochloric acid at room temperature and were vortexed for 1 min. 26 mL of cold (around −20° C.) concentrated acid was added to the mixture and vortexed for 1 min. The mixture composed of 2 g/L of biomass in aqueous HCl was then heated up to room temperature. At this time, hydrolysis time was set to 0. The mixture was placed in a steering wheel for 45 min. The mixture was subsequently centrifuged (4000 RPM, rt, 15 min), and the supernatant was filtered. This step corresponded to the extraction of insoluble lignin from the hydrolysate, and lasted 5-10 min.

During this procedure, samples were taken to determine xylose, arabinose and glucose concentrations. Samples were analyzed by HPLC Shimadzu after dilution in water. The kinetic plot is provided in Table 19. It should be understood that 48 hours was considered to be the time of full hydrolysis, which was used to determine the total amount of C5 and C6 sugars.

TABLE 19
Release of glucose and xylose monomers versus time
relative to final release after 48 H, during treatment of
miscanthus with concentrated aqueous HCl at room temperature

| Time of | Xylose- | |
|---|---|---|
| hydrolysis | Arabinose | Gucose |
| min | % | % |
| 0 | 0 | 0 |
| 15 | 54% | 4% |
| 30 | 78% | 3% |
| 60 | 101% | 3% |
| 120 | 104% | 8% |

This data showed that after 1 hour of hydrolysis and lignin filtration, the composition of the hydrolysate was 4% of glucose, 96% of glucan, 100% of xylose and 0% of xylan. These percentages as well as percentages in Table 19 above represent fraction of the total C5/C6 sugars present in the stated form.

B. Selective Adsorption of C6 Versus C5

A raw biomass hydrolysate was prepared following the procedure described in Example 19A above, with a targeted biomass concentration of 10 g/L in concentrated aqueous HCl (37%). In addition to the protocol described in Example 19A above, a step of adsorption on MCN was included after completing the lignin extraction. 40 mg of MCN were used for 10 mL of hydrolysate.

The analysis of the hydrolysate after full hydrolysis showed that only C6 carbohydrate was adsorbed on MCN. The fraction of C6 sugars adsorbed relative to total sugar content adsorbed was nearly 100% under these conditions, and was typically loaded with around 200 mg of adsorbed long-chain glucan per gram of MCN.

Xylose, arabinose and glucose concentrations were analyzed by HPLC Shimadzu after dilution in water. The result of the selective adsorption of C6 is shown in Table 20 below.

TABLE 20
Selective adsorption of C6 versus C5.

| | Glu (C6) | Xyl (C5) |
|---|---|---|
| | g/L | g/L |
| Hydrolysate Content Without Adsorption | 3.38 | 1.52 |
| Hydrolysate Content With Adsorption on MCN | 2.54 | 1.52 |
| Concentration adsorbed | 0.84 | 0.00 |
| % of sugars originally present in solution that is | 25% | 0% |
| adsorbed | | |

C. Effect of Selectivity and MCN Loading on Adsorption of Glucan on the Surface of MCN

This example demonstrates nearly quantitative glucan adsorption from concentrated acid hydrolysate, which was accomplished by using an excess of MCN.

A raw biomass hydrolysate was prepared following the procedure described in Example 19A above, with a targeted biomass concentration of 10 g/L in concentrated aqueous HCl (37%). Additionally to the protocol of Example 19A above, a step of adsorption on MCN was included after completing lignin extraction. 15 mg, 20 mg or 32 mg of MCN was used for 1 mL of hydrolysate Aiming for a quantitative adsorption of glucan, MCN was used in slight excess compared with the glucan amount to be adsorbed.

Glucose, xylose and arabinose concentration were analyzed by HPLC Shimadzu after dilution in water. The selectivity, here defined by the mass ratio of glucan adsorbed/xylose adsorbed, was presented in Table 21 below.

TABLE 21
Adsorption of carbohydrate on MCN, Selectivity of glucan
adsorption (glucose polymer) versus xylose monomers adsorption

| | Mass selectivity |
|---|---|
| Mass MCN | m gluc. eq. adsorbed/m |
| mg | xylose adsorbed |
| 15 | 31.1 |
| 20.4 | 37.8 |
| 32.7 | 20.3 |

Including data previously obtained in Example 19B above, Table 22 below summarizes data of the percentage of xylose adsorption from concentrated acid hydrolysate solution versus the percentage of glucan adsorption from concentrated acid hydrolysate solution. The carbon material loading obtained ranges from 75 to 210 mgGlueq./gMCN, and is labeled near each point.

TABLE 22
Percentage of xylose adsorption on MCN vs.
percentage of glucan adsorption on MCN

| | Glucan | Xylose |
|---|---|---|
| | Glucan | C5 |
| [Glu]eq. | adsorbed | adsorbed |
| mgGlueq./gMCN | % | % |
| 145 | 86% | 4% |
| 111 | 89% | 4% |
| 75 | 96% | 7% |
| 190 | 75% | 0% |
| 210 | 25% | 0% |

1-127. (canceled)

128. A method of separating at least one oligomer from a mixture of oligomers, comprising contacting the mixture of oligomers with carbonaceous material at a pH of about 4.0 or below under conditions whereby the carbonaceous material adsorbs at least one oligomer from the mixture of oligomers to separate the at least one oligomer,
wherein the carbonaceous material is activated charcoal, activated coal, activated carbon, powdered activated carbon, granular activated carbon, extruded activated carbon, bead activated carbon, impregnated carbon, polymer coated carbon, or mesoporous carbon material.

129. The method of claim 128, wherein the carbonaceous material is mesoporous carbon material.

130. The method of claim 129, wherein the mesoporous carbon material is a mesoporous carbon nanoparticle.

131. The method of claim 130, wherein the mesoporous carbon nanoparticle is a CMK-1 type nanoparticle, a CMK-3 type nanoparticle, a CMK-5 type nanoparticle, or a CMK-8 type nanoparticle.

132. The method of claim 130, wherein the mesoporous carbon nanoparticle has an average particle size that ranges from about 10 nm to about 200 nm.

133. The method of claim 128, wherein the carbonaceous material has a surface area of at least about 1000 m²/g.

134. The method of claim 133, wherein the carbonaceous material has a surface area between about 1000 m²/g and about 3000 m²/g.

135. The method of claim 129, wherein the mesoporous carbon material further comprises an acid-functionalized surface, a base-functionalized surface, or an acid/base-functionalized surface.

136. The method of claim 128, further comprising depolymerizing biomass to produce the mixture of oligomers.

137. The method of claim 136, wherein the depolymerizing is performed by acid hydrolysis.

138. The method of claim 128, wherein the mixture of oligomers comprises lignin oligomers, hemicellulose oligomers, or any combinations thereof.

139. The method of claim 128, wherein the mixture of oligomers comprises C5 oligomers and C6 oligomers.

140. The method of claim 128, wherein the mixture of oligomers comprises xylose, xylans, xyloglucans, mannans, mannose, galactose, rhamnose, arabinose, or arabinoxylans, or any combinations thereof.

141. The method of claim 128, wherein the mixture of oligomers comprises poly-β-glucan fragments or long chain poly-β-glucans.

142. The method of claim 141, wherein the poly-β-glucan fragments comprise glucose, cellobiose, cellotriose, cellotetraose, cellopentaose, or cellohexaose, or any combinations thereof.

143. The method of claim 128, further comprising desorbing at least one adsorbed oligomer from the carbonaceous material.

144. The method of claim 142, wherein the desorbing is performed using ionic liquid, acid, ethanol, water, a mixture of ethanol and water, or a mixture of LiCl and N,N-dimethylacetamide.

145. The method of claim 143, wherein the desorbing is performed using acid, wherein the acid is HCl, H₃PO₃, H₃PO₄, H₂SO₄, H₂SO₃, HF, HlO₄, HBr, H₃O⁺, HNO₂, HNO₃, HI, boronic acid, or polyoxometallate acid, or any combinations thereof.

146. The method of claim 128, further comprising depolymerizing at least one adsorbed oligomer into at least one shorter chain oligomer.

147. The method of claim 146, wherein at least one shorter chain oligomer is a monosaccharide.

148. The method of claim 146, wherein at least one shorter chain oligomer is glucose, xylose, or a combination thereof.
